(19) United States
(12) Patent Application Publication
(10) Pub. No.: US 2002/0076715 A1
(43) Pub. Date: Jun. 20, 2002

(54) Compositions and methods for ovarian cancer therapy and diagnosis

(76) Inventors: Darin R. Benson, Seattle, WA (US); Michael J. Lodes, Seattle, WA (US); Jennifer L. Mitcham, Redmond, WA (US); Gordon E. King, Shoreline, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/876,889

(22) Filed: Jun. 6, 2001

Related U.S. Application Data
(63) Continuation-in-part of application No. 09/397,787, filed on Sep. 16, 1999.
Application No. 09/397,787 is a continuation-in-part of application No. 09/246,429, filed on Feb. 8, 1999, now abandoned.
Application No. 09/246,429 is a continuation-in-part of application No. 09/159,320, filed on Sep. 23, 1998, now abandoned.

(51) Int. Cl. C12Q 1/68; C07H 21/04; G06F 19/00; G01N 33/48; G01N 33/50

(52) U.S. Cl. 435/6; 702/20; 536/24.3

(57) ABSTRACT

Compositions and methods for the therapy and diagnosis of cancer, such as ovarian cancer, are disclosed. Compositions may comprise one or more ovarian carcinoma proteins, immunogenic portions thereof, polynucleotides that encode such portions or antibodies or immune system cells specific for such proteins. Such compositions may be used, for example, for the prevention and treatment of diseases such as ovarian cancer. Methods are further provided for identifying tumor antigens that are secreted from ovarian carcinomas and/or other tumors. Polypeptides and polynucleotides as provided herein may further be used for the diagnosis and monitoring of ovarian cancer.

### CROSS-REFERENCE TO RELATED APPLICATIONS

This application is a CIP of U.S. application Ser. No. 09/397,787 filed Sep. 16, 1999 (pend.), which is a CIP of U.S. application Ser. No. 09/246,429 filed Feb. 8, 1999 (ab.), which is a CIP of U.S. application Ser. No. 09/159,320 filed Sep. 23, 1998 (ab.), all incorporated in their entirety herein by reference.

### TECHNICAL FIELD

The present invention relates generally to ovarian cancer therapy. The invention is more specifically related to polypeptides comprising at least a portion of an ovarian carcinoma protein, and to polynucleotides encoding such polypeptides, as well as antibodies and immune system cells that specifically recognize such polypeptides. Such polypeptides, polynucleotides, antibodies and cells may be used in vaccines and pharmaceutical compositions for treatment of ovarian cancer.

### BACKGROUND OF THE INVENTION

Ovarian cancer is a significant health problem for women in the United States and throughout the world. Although advances have been made in detection and therapy of this cancer, no vaccine or other universally successful method for prevention or treatment is currently available. Management of the disease currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and high mortality continues to be observed in many cancer patients.

Immunotherapies have the potential to substantially improve cancer treatment and survival. Such therapies may involve the generation or enhancement of an immune response to an ovarian carcinoma antigen. However, to date, relatively few ovarian carcinoma antigens are known and the generation of an immune response against such antigens has not been shown to be therapeutically beneficial.

Accordingly, there is a need in the art for improved methods for identifying ovarian tumor antigens and for using such antigens in the therapy of ovarian cancer. The present invention fulfills these needs and further provides other related advantages.

### SUMMARY OF THE INVENTION

Briefly stated, this invention provides compositions and methods for the therapy of cancer, such as ovarian cancer. In one aspect, the present invention provides polypeptides comprising an immunogenic portion of an ovarian carcinoma protein, or a variant thereof that differs in one or more substitutions, deletions, additions and/or insertions such that the ability of the variant to react with ovarian carcinoma protein-specific antisera is not substantially diminished. Within certain embodiments, the ovarian carcinoma protein comprises a sequence that is encoded by a polynucleotide sequence selected from the group consisting of sequences recited in FIGS. 5, 8, 13, 16A-16I and 17A-17C (SEQ ID NO:5, 8, 13, 19-68 and 69-85), SEQ ID NO:130, 133, 135, 137, 138, 140, 142, 144-146, 148, 153, 155, 158, 159, 161, 163, 170, 174, 177, 178, 179, 180-182, 184, 187, 189, 190, 192, 195, 196, 198, 200, 207, 214-216, 220, 221, 222, 227, 228, 230, 231, 232, 234, 240-242, 243, 244-246, 250, 251, 253, 254, 256, 259, 261, 262, 264, 265, 272-275, 276, 278, 280, 281, 282, 283, 287, 288, 291, 292, 296, 297, 299, 301, 304-309, 311, 313, 314, 317, 323, 325 333, and 335-345 and complements of such polynucleotides.

The present invention further provides polynucleotides that encode a polypeptide as described above or a portion thereof, expression vectors comprising such polynucleotides and host cells transformed or transfected with such expression vectors.

Within other aspects, the present invention provides pharmaceutical compositions and vaccines. Pharmaceutical compositions may comprise a physiologically acceptable carrier in combination with one or more of: (i) a polypeptide comprising an immunogenic portion of an ovarian carcinoma protein, or a variant thereof that differs in one or more substitutions, deletions, additions and/or insertions such that the ability of the variant to react with ovarian carcinoma protein-specific antisera is not substantially diminished, wherein the ovarian carcinoma protein comprises an amino acid sequence encoded by a polynucleotide that comprises a sequence recited in any one of FIGS. 5, 8, 13, 16A-16I and 17A-17C (SEQ ID NO: 5, 8, 13, 19-68 and 69-85), SEQ ID NO:130, 133, 135, 137, 138, 140, 142, 144-146, 148, 153, 155, 158, 159, 161, 163, 170, 174, 177, 178, 179, 180-182, 184, 187, 189, 190, 192, 195, 196, 198, 200, 207, 214-216, 220, 221, 222, 227, 228, 230, 231, 232, 234, 240-242, 243, 244-246, 250, 251, 253, 254, 256, 259, 261, 262, 264, 265, 272-275, 276, 278, 280, 281, 282, 283, 287, 288, 291, 292, 296, 297, 299, 301, 304-309, 311, 313, 314, 317, 323, 325, 333, or 335-345 or a complement of any of the foregoing sequences; (ii) a polynucleotide encoding at least 5 amino acid residues of such an ovarian carcinoma protein; (iii) an antibody that specifically binds to such an ovarian carcinoma protein; (iv) an antigen-presenting cell that expresses a polypeptide as described above and/or (v) a T cell that specifically reacts with such a polypeptide. Vaccines may comprise a non-specific immune response enhancer in combination with one or more of: (i) a polypeptide comprising an immunogenic portion of an ovarian carcinoma protein, or a variant thereof that differs in one or more substitutions, deletions, additions and/or insertions such that the ability of the variant to react with ovarian carcinoma protein-specific antisera is not substantially diminished, wherein the ovarian carcinoma protein comprises an amino acid sequence encoded by a polynucleotide that comprises a sequence recited in any one of FIGS. 5, 8, 13, 16A-16I and 17A-17C (SEQ ID NO: 5,8,13,19-68 and 69-85), SEQ ID NO:130, 133, 135, 137, 138, 140, 142, 144-146, 148, 153, 155, 158, 159, 161, 163, 170, 174, 177, 178, 179, 180-182, 184, 187, 189, 190, 192, 195, 196, 198, 200, 207, 214-216, 220, 221, 222, 227, 228, 230, 231, 232, 234, 240-242, 243, 244-246, 250, 251, 253, 254, 256, 259, 261, 262, 264, 265, 272-275, 276, 278, 280, 281, 282, 283, 287, 288, 291, 292, 296, 297, 299, 301, 304-309, 311, 313, 314, 317, 323, 325, 333, or 335-345 or a complement of any of the foregoing sequences; (ii) a polynucleotide encoding at least 5 amino acid residues of such an ovarian carcinoma protein; (iii) an anti-idiotypic antibody that is specifically bound by an antibody that specifically binds to such a polypeptide; (iv) an antigen-presenting cell that expresses such a polypeptide and/or (v) a T cell that specifically reacts with such a polypeptide.

The present invention further provides, in other aspects, fusion proteins that comprise at least one polypeptide as described above, as well as polynucleotides encoding such fusion proteins.

Within related aspects, pharmaceutical compositions comprising a fusion protein or polynucleotide encoding a fusion protein in combination with a physiologically acceptable carrier are provided.

Vaccines are further provided, within other aspects, comprising a fusion protein or polynucleotide encoding a fusion protein in combination with a non-specific immune response enhancer.

Within further aspects, the present invention provides methods for inhibiting the development of a cancer in a patient, comprising administering to a patient a pharmaceutical composition or vaccine as recited above.

The present invention further provides, within other aspects, methods for stimulating and/or expanding T cells, comprising contacting T cells with (a) a polypeptide comprising an immunogenic portion of an ovarian carcinoma protein, or a variant thereof that differs in one or more substitutions, deletions, additions and/or insertions such that the ability of the variant to react with ovarian carcinoma protein-specific antisera is not substantially diminished, wherein the ovarian carcinoma protein comprises an amino acid sequence encoded by a polynucleotide that comprises a sequence recited in any one of SEQ ID NO:1-334; (b) a polynucleotide encoding such a polypeptide and/or (c) an antigen presenting cell that expresses such a polypeptide under conditions and for a time sufficient to permit the stimulation and/or expansion of T cells. Such polypeptide, polynucleotide and/or antigen presenting cell(s) may be present within a pharmaceutical composition or vaccine, for use in stimulating and/or expanding T cells in a mammal.

Within other aspects, the present invention provides methods for inhibiting the development of ovarian cancer in a patient, comprising administering to a patient T cells prepared as described above.

Within further aspects, the present invention provides methods for inhibiting the development of ovarian cancer in a patient, comprising the steps of: (a) incubating CD4⁺ and/or CD8⁺ T cells isolated from a patient with one or more of: (i) a polypeptide comprising an immunogenic portion of an ovarian carcinoma protein, or a variant thereof that differs in one or more substitutions, deletions, additions and/or insertions such that the ability of the variant to react with ovarian carcinoma protein-specific antisera is not substantially diminished, wherein the ovarian carcinoma protein comprises an amino acid sequence encoded by a polynucleotide that comprises a sequence recited in any one of SEQ ID NO:1-345; (ii) a polynucleotide encoding such a polypeptide; or (iii) an antigen-presenting cell that expresses such a polypeptide; such that T cells proliferate; and (b) administering to the patient an effective amount of the proliferated T cells, and thereby inhibiting the development of ovarian cancer in the patient. The proliferated cells may be cloned prior to administration to the patient.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE SEQUENCE IDENTIFIERS

SEQ ID NO:1-4, 6-7, 9-12, and 14-15 are described in Table I. Clones containing these sequences were derived from a cDNA library constructed using sera obtained from patients with ovarian cancer.

SEQ ID NO:5, 8, and 13 are partial nucleotide sequences derived from the clones OV10, OV17, and OV41 respectively.

SEQ ID NO: 16 is the amino acid sequence of the encoded polypeptide of SEQ ID NO:1, which depicts a partial sequence of a polynucleotide encoding a ovarian carcinoma antigen designated OV2.

SEQ ID NO:17 is the amino acid sequence of the encoded polypeptide of SEQ ID NO:2, which depicts a partial sequence of a polynucleotide encoding a ovarian carcinoma antigen designated OV3.

SEQ ID NO:18 is the amino acid sequence of the encoded polypeptide of SEQ ID NO:3, which depicts a partial sequence of a polynucleotide encoding a ovarian carcinoma antigen designated OV6.

SEQ ID NO:19-129 are described in Table II and depict partial sequences of polynucleotides encoding further representative ovarian carcinoma antigens.

SEQ ID NO:130-334 are described in Table III and depict partial sequences of polynucleotides encoding further representative ovarian carcinoma antigens.

SEQ ID NO:335 depicts the full length polynucleotide sequence of clone 055E/OVp2-1.

SEQ ID NO:336 depicts the full length polynucleotide sequence of clone 056E/OVp2-2.

SEQ ID NO:337 depicts the full length polynucleotide sequence of clone 057E/OVp2-4.

SEQ ID NO:338 depicts the full length polynucleotide sequence of clone 058E/OVp2-56.

SEQ ID NO:339 depicts the full length polynucleotide sequence of clone 053E/OVp2-30.

SEQ ID NO:340 depicts the full length polynucleotide sequence of clone 059E/OVp2-88.

SEQ ID NO:341 depicts the full length polynucleotide sequence of clone 060E/OVp2-94.

SEQ ID NO:342 depicts the full length polynucleotide sequence of clone OVp2-28.

SEQ ID NO:343 depicts the full length polynucleotide sequence of clone OVp2-244.

SEQ ID NO:344 depicts the full length polynucleotide sequence of clone OVp2-285.

SEQ ID NO:345 depicts the full length polynucleotide sequence of clone OVp4-2.

SEQ ID NO:346 depicts the full length amino acid sequence of clone 055E/OVp2-1.

SEQ ID NO:347 depicts the full length amino acid sequence of clone 057E/OVp2-4.

SEQ ID NO:348 depicts the full length amino acid sequence of clone 058E/OVp2-56.

SEQ ID NO:349 depicts the full length amino acid sequence of clone 059E/OVp2-88.

SEQ ID NO:350 depicts the full length amino acid sequence of clone 060E/OVp2-94.

SEQ ID NO:351 depicts the full length amino acid sequence of clone OVp2-28.

SEQ ID NO:352 depicts the full length amino acid sequence of clone OVp2-244.

SEQ ID NO:353 depicts the full length amino acid sequence of clone OVp4-2.

### BRIEF DESCRIPTION OF THE DRAWINGS

FIG. 1 depicts a partial sequence of a polynucleotide encoding a ovarian carcinoma antigen designated OV2 (SEQ ID NO:1), and the amino acid sequence of the encoded polypeptide (SEQ ID NO:16).

FIG. 2 depicts a partial sequence of a polynucleotide encoding a ovarian carcinoma antigen designated OV3 (SEQ ID NO:2), and the amino acid sequence of the encoded polypeptide (SEQ ID NO:17).

FIG. 3 depicts a partial sequence of a polynucleotide encoding a ovarian carcinoma antigen designated OV6 (SEQ ID NO:3), and the amino acid sequence of the encoded polypeptide (SEQ ID NO: 18).

FIG. 4 depicts a partial sequence of a polynucleotide encoding a ovarian carcinoma antigen designated OV9 (SEQ ID NO:4).

FIG. 5 depicts a partial sequence of a polynucleotide encoding a ovarian carcinoma antigen designated OV10(SEQID NO:5).

FIG. 6 depicts a partial sequence of a polynucleotide encoding a ovarian carcinoma antigen designated OV12 (SEQ ID NO:6).

FIG. 7 depicts a partial sequence of a polynucleotide encoding a ovarian carcinoma antigen designated OV14 (SEQ ID NO:7).

FIG. 8 depicts a partial sequence of a polynucleotide encoding a ovarian carcinoma antigen designated OV17 (SEQ ID NO:8).

FIG. 9 depicts a partial sequence of a polynucleotide encoding a ovarian carcinoma antigen designated OV18 (SEQ ID NO:9).

FIG. 10 depicts a partial sequence of a polynucleotide encoding a ovarian carcinoma antigen designated OV23 (SEQ ID NO:10).

FIG. 11 depicts a partial sequence of a polynucleotide encoding a ovarian carcinoma antigen designated OV24 (SEQ ID NO:11).

FIG. 12 depicts a partial sequence of a polynucleotide encoding a ovarian carcinoma antigen designated OV27 (SEQ ID NO:12).

FIG. 13 depicts a partial sequence of a polynucleotide encoding a ovarian carcinoma antigen designated OV41 (SEQ ID NO:13).

FIG. 14 depicts a partial sequence of a polynucleotide encoding a ovarian carcinoma antigen designated OV54 (SEQ ID NO:14).

FIG. 15 depicts a partial sequence of a polynucleotide encoding a ovarian carcinoma antigen designated OV57 (SEQ ID NO:15).

FIGS. 16A-16I depict partial sequences of polynucleotides (SEQ ID NO: 19-68) encoding further representative ovarian carcinoma antigens.

FIGS. 17A-17C depict partial sequences of polynucleotides (SEQ ID NO: 69-85) encoding further representative ovarian carcinoma antigens.

FIGS. 18A-18F depict partial sequences of polynucleotides (SEQ ID NO: 86-129) encoding further representative ovarian carcinoma antigens.

### DETAILED DESCRIPTION OF THE INVENTION

As noted above, the present invention is generally directed to compositions and methods for the detection and therapy of cancer, such as ovarian cancer. The compositions described herein may include immunogenic polypeptides, polynucleotides encoding such polypeptides, binding agents such as antibodies that bind to a polypeptide, antigen presenting cells (APCs) and/or immune system cells (e.g., T cells).

Polypeptides of the present invention generally comprise at least an immunogenic portion of an ovarian carcinoma protein or a variant thereof. Certain ovarian carcinoma proteins have been identified using an immunoassay technique, and are referred to herein as ovarian carcinoma antigens. An “ovarian carcinoma antigen” is a protein that is expressed by ovarian tumor cells (preferably human cells) at a level that is at least two fold higher than the level in normal ovarian cells. Certain ovarian carcinoma antigens react detectably (within an immunoassay, such as an ELISA or Western blot) with antisera generated against serum from an immunodeficient animal implanted with a human ovarian tumor. Such ovarian carcinoma antigens are shed or secreted from an ovarian tumor into the sera of the immunodeficient animal. Accordingly, certain ovarian carcinoma antigens provided herein are secreted antigens. Certain nucleic acid sequences of the subject invention generally comprise a DNA or RNA sequence that encodes all or a portion of such a polypeptide, or that is complementary to such a sequence.

The present invention further provides ovarian carcinoma sequences that are identified using techniques to evaluate altered expression within an ovarian tumor. Such sequences may be polynucleotide or protein sequences. Ovarian carcinoma sequences are generally expressed in an ovarian tumor at a level that is at least two fold, and preferably at least five fold, greater than the level of expression in normal ovarian tissue, as determined using a representative assay provided herein. Certain partial ovarian carcinoma polynucleotide sequences are presented herein. Proteins encoded by genes comprising such polynucleotide sequences (or complements thereof) are also considered ovarian carcinoma proteins.

Antibodies are generally immune system proteins, or antigen-binding fragments thereof, that are capable of binding to at least a portion of an ovarian carcinoma polypeptide as described herein. T cells that may be employed within the compositions provided herein are generally T cells (e.g., CD4⁺ and/or CD8⁺) that are specific for such a polypeptide. Certain methods described herein further employ antigen-presenting cells (such as dendritic cells or macrophages) that express an ovarian carcinoma polypeptide as provided herein.

Ovarian Carcinoma Polynucleotides

Any polynucleotide that encodes an ovarian carcinoma protein or a portion or other variant thereof as described herein is encompassed by the present invention. Preferred polynucleotides comprise at least 15 consecutive nucleotides, preferably at least 30 consecutive nucleotides, and more preferably at least 45 consecutive nucleotides, that encode a portion of an ovarian carcinoma protein. More preferably, a polynucleotide encodes an immunogenic portion of an ovarian carcinoma protein, such as an ovarian carcinoma antigen. Polynucleotides complementary to any such sequences are also encompassed by the present invention. Polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an ovarian carcinoma protein or a portion thereof) or may comprise a variant of such a sequence. Polynucleotide variants may contain one or more substitutions, additions, deletions and/or insertions such that the immunogenicity of the encoded polypeptide is not diminished, relative to a native ovarian carcinoma protein. The effect on the immunogenicity of the encoded polypeptide may generally be assessed as described herein. Variants preferably exhibit at least about 70% identity, more preferably at least about 80% identity and most preferably at least about 90% identity to a polynucleotide sequence that encodes a native ovarian carcinoma protein or a portion thereof.

The percent identity for two polynucleotide or polypeptide sequences may be readily determined by comparing sequences using computer algorithms well known to those of ordinary skill in the art, such as Megalign, using default parameters. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned. Optimal alignment of sequences for comparison may be conducted, for example, using the Megalign program in the Lasergene suite of bioinformatics software (DNASTAR, Inc., Madison, Wis.), using default parameters. Preferably, the percentage of sequence identity is determined by comparing two optimally aligned sequences over a window of comparison of at least 20 positions, wherein the portion of the polynucleotide or polypeptide sequence in the window may comprise additions or deletions (i.e., gaps) of 20% or less, usually 5 to 15%, or 10 to 12%, relative to the reference sequence (which does not contain additions or deletions). The percent identity may be calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native ovarian carcinoma protein (or a complementary sequence). Suitable moderately stringent conditions include prewashing in a solution of 5× SSC, 0.5% SDS, 1.0 mM EDTA (pH 8.0); hybridizing at 50° C.-65° C., 5× SSC, overnight; followed by washing twice at 65° C. for 20 minutes with each of 2×, 0.5× and 0.2× SSC containing 0.1% SDS.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Polynucleotides may be prepared using any of a variety of techniques. For example, an ovarian carcinoma polynucleotide may be identified, as described in more detail below, by screening a late passage ovarian tumor expression library with antisera generated against sera of immunocompetent mice after injection of such mice with sera from SCID mice implanted with late passage ovarian tumors. Ovarian carcinoma polynucleotides may also be identified using any of a variety of techniques designed to evaluate differential gene expression. Alternatively, polynucleotides may be amplified from cDNA prepared from ovarian tumor cells. Such polynucleotides may be amplified via polymerase chain reaction (PCR). For this approach, sequence-specific primers may be designed based on the sequences provided herein, and may be purchased or synthesized.

An amplified portion may be used to isolate a full length gene from a suitable library (e.g., an ovarian carcinoma cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences are then assembled into a single contiguous sequence. A full length cDNA molecule can be generated by ligating suitable fragments, using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence.

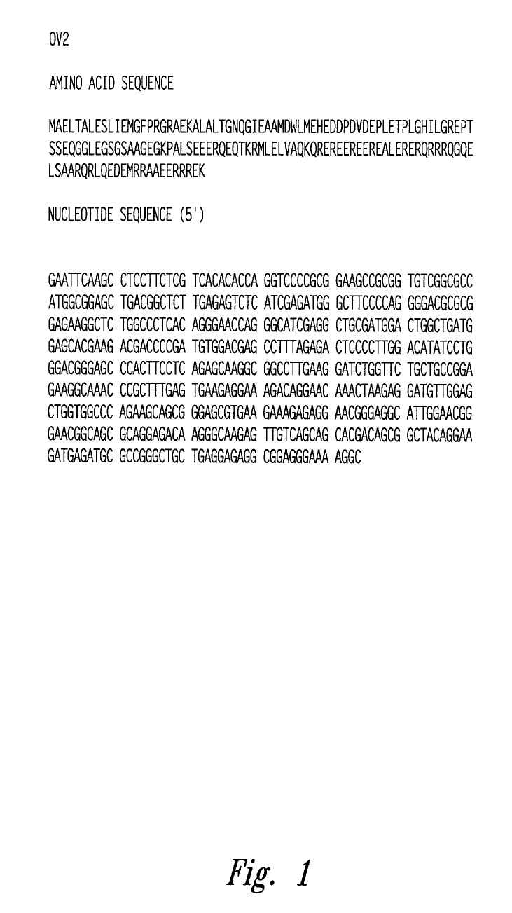

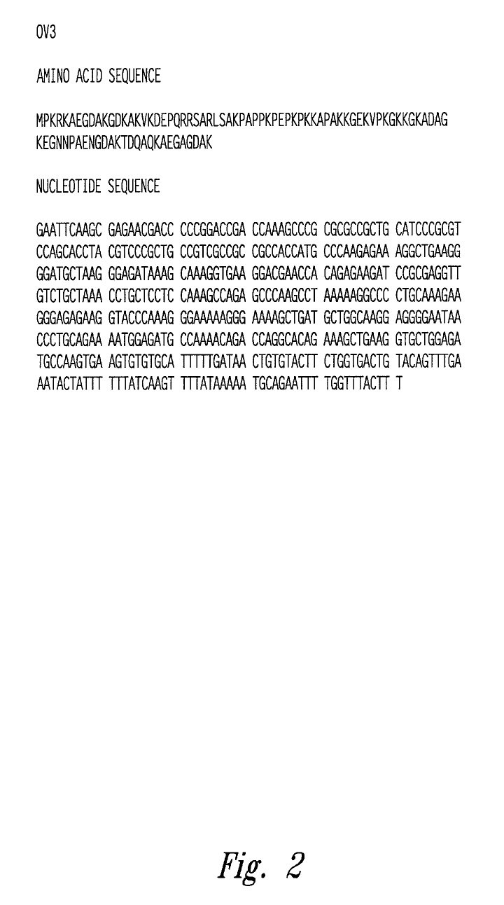

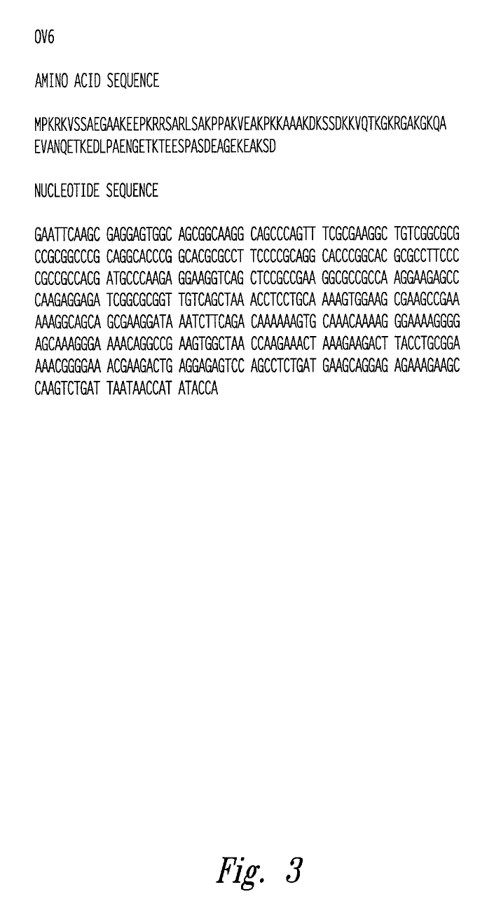

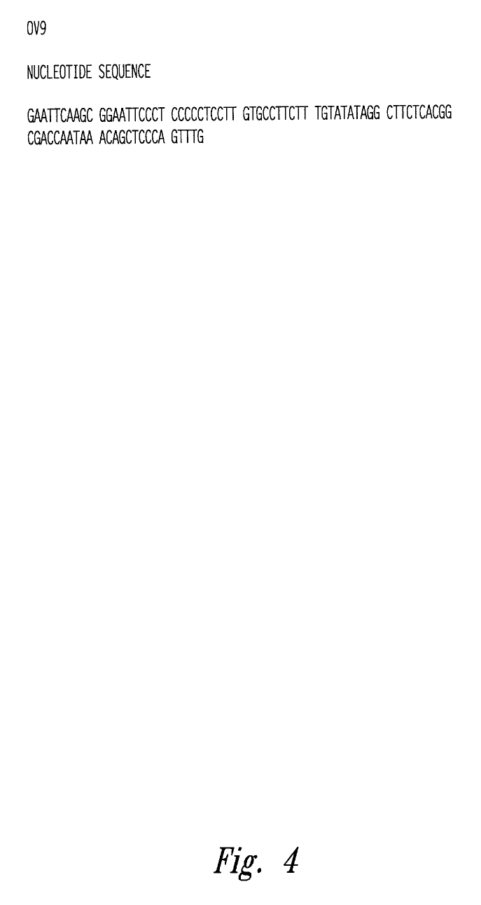

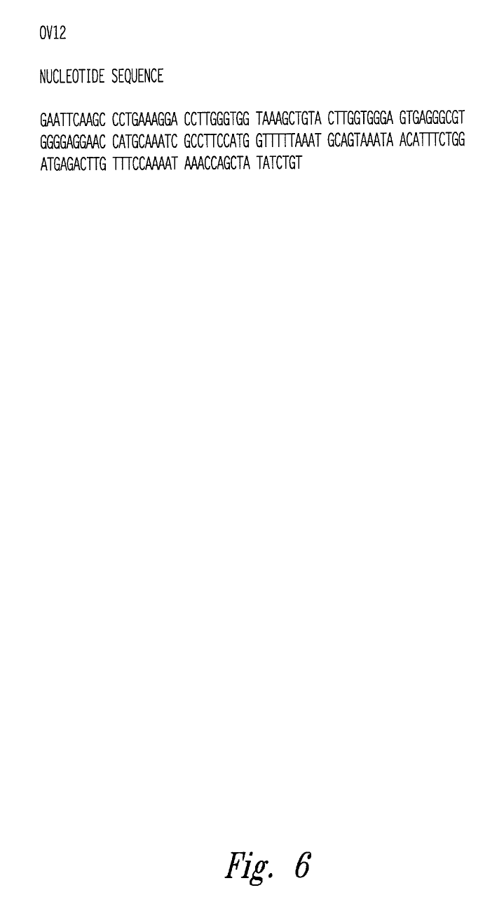

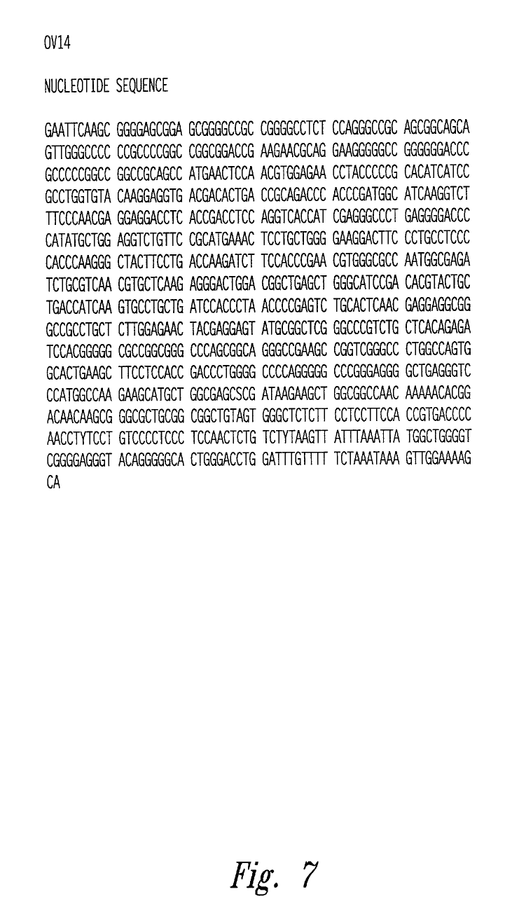

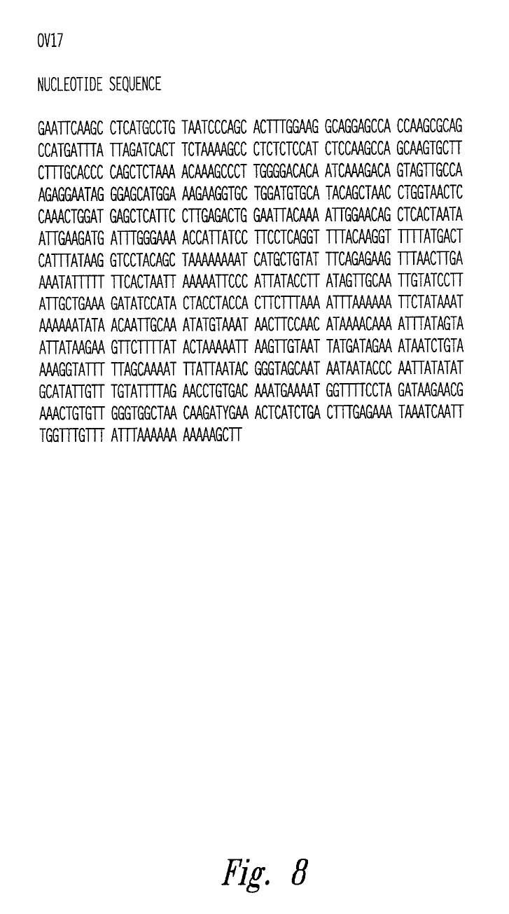

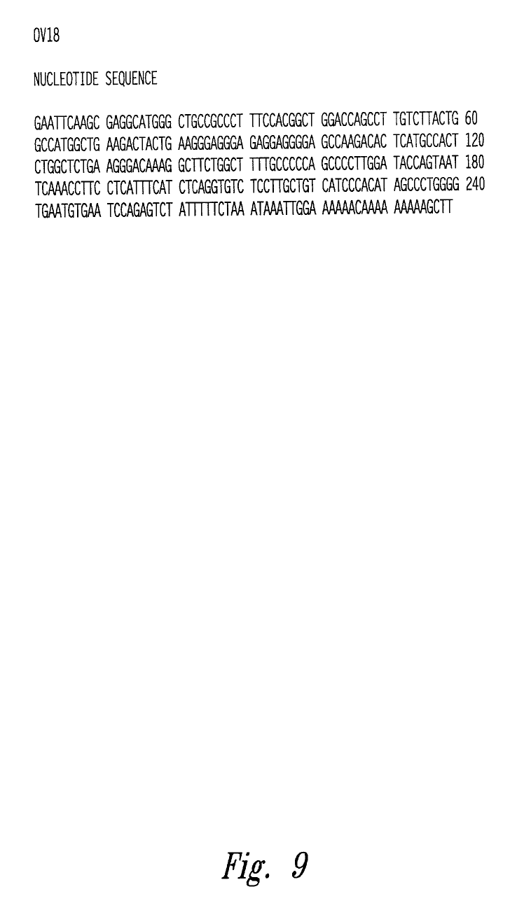

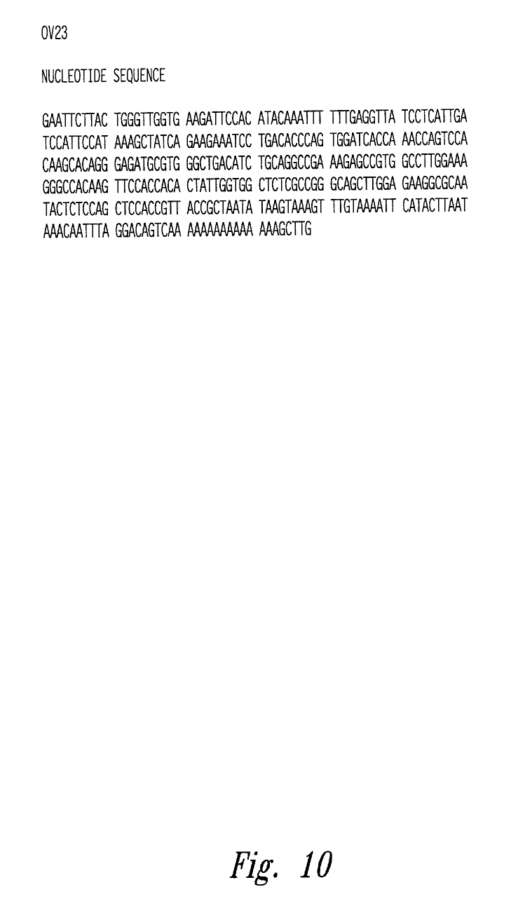

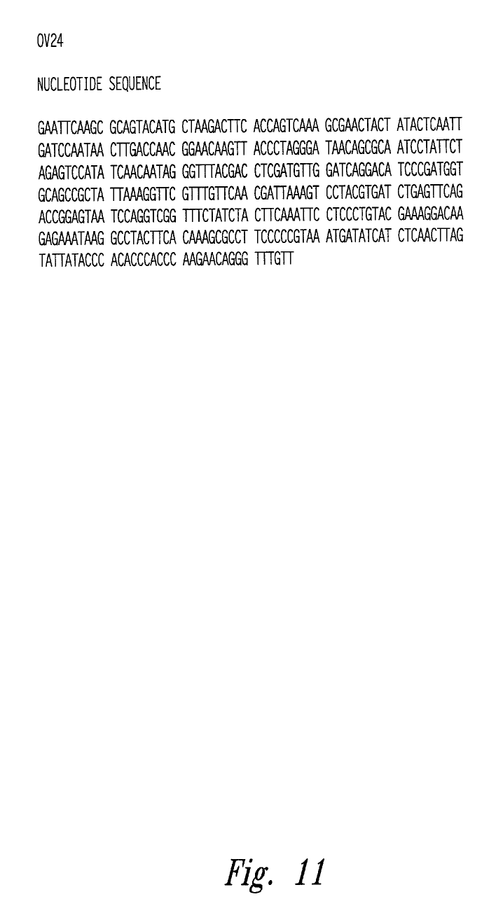

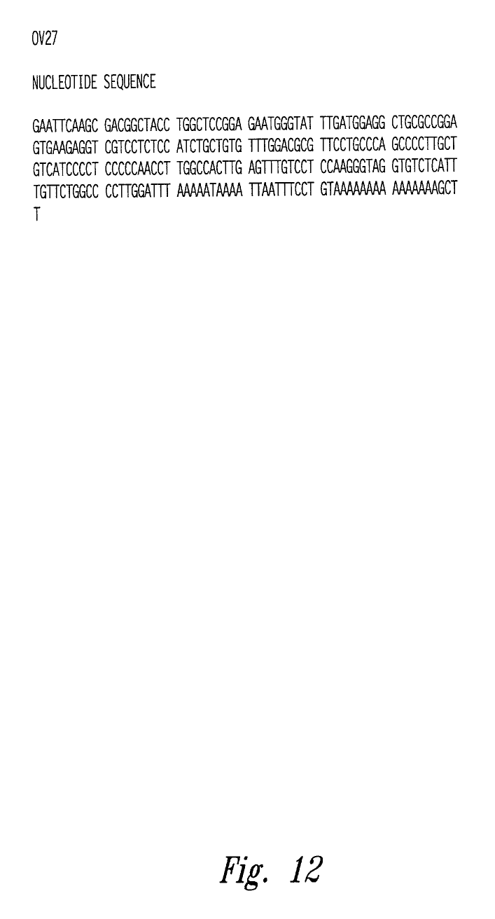

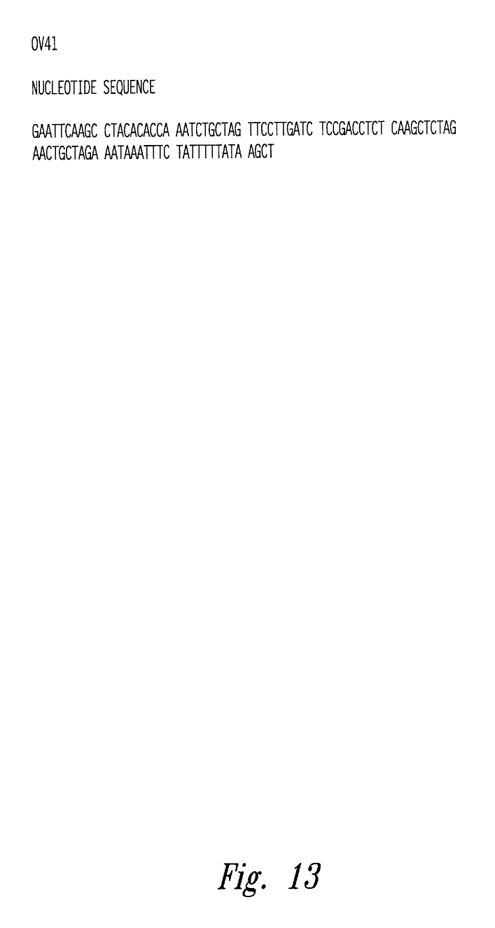

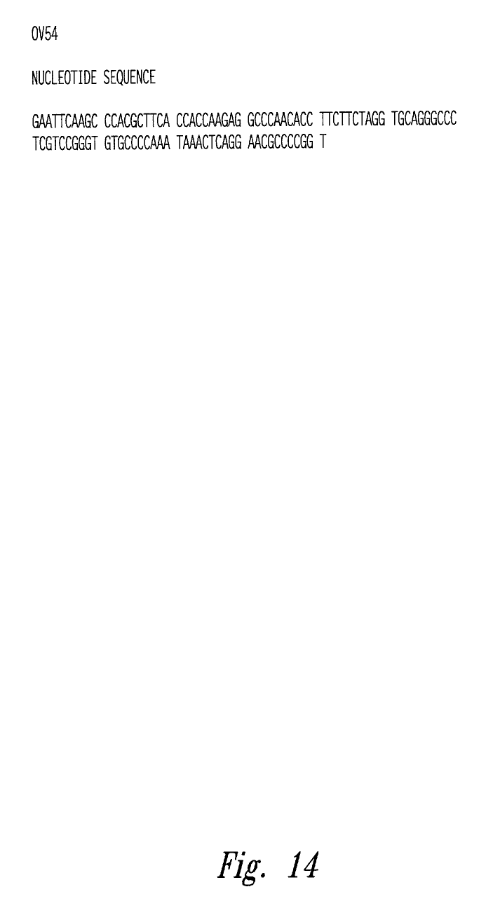

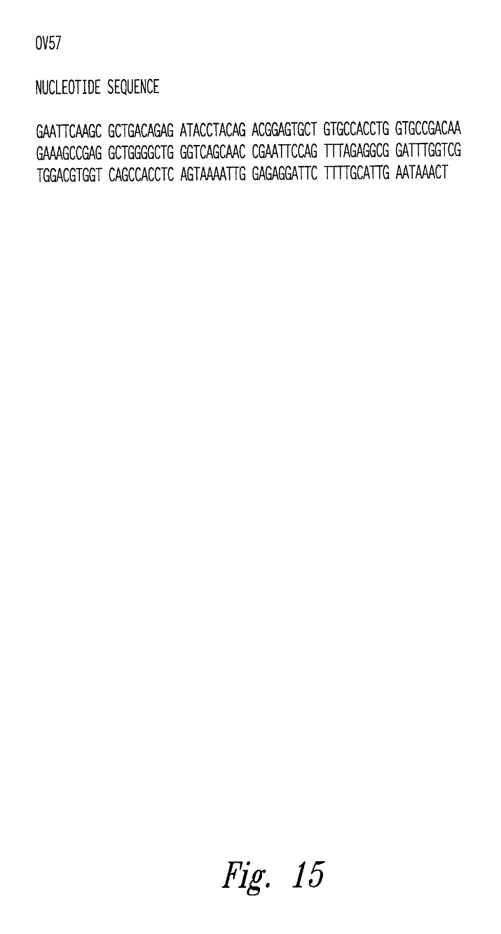

Certain nucleic acid sequences of cDNA molecules encoding portions of ovarian carcinoma proteins are provided in FIGS. 1-15, 16A-16I, 17A-17C and 18A-18F (SEQ ID NO:1-15, 19-68, 69-85 and 86-129). Other such nucleic acid sequences are provided in SEQ ID NO:130-334. These polynucleotides were isolated by serological screening of an ovarian tumor cDNA expression library. The library was prepared from unamplified cDNA derived from a human ovarian tumor OV9334 grown in a SCID mouse, in the vector pScreen. Sera from human patients with ovarian cancer were pooled for the screen.

The polynucleotides recited herein, as well as full length polynucleotides comprising such sequences, other portions of such full length polynucleotides, and sequences complementary to all or a portion of such full length molecules, are specifically encompassed by the present invention. It will be apparent to those of ordinary skill in the art that this technique can also be applied to the identification of antigens that are secreted from other types of tumors.

Any of a variety of well known techniques may be used to evaluate tumor-associated expression of a cDNA. For example, hybridization techniques using labeled polynucleotide probes may be employed. Alternatively, or in addition, amplification techniques such as real-time PCR may be used (see Gibson et al., Genome Research 6:995-1001, 1996; Heid et al., Genome Research 6:986-994, 1996). Real-time PCR is a technique that evaluates the level of PCR product accumulation during amplification. This technique permits quantitative evaluation of mRNA levels in multiple samples. Briefly, mRNA is extracted from tumor and normal tissue and cDNA is prepared using standard techniques. Real-time PCR may be performed, for example, using a Perkin Elmer/Applied Biosystems (Foster City, Calif.) 7700 Prism instrument. Matching primers and fluorescent probes may be designed for genes of interest using, for example, the primer express program provided by Perkin Elmer/Applied Biosystems (Foster City, Calif.). Optimal concentrations of primers and probes may be initially determined by those of ordinary skill in the art, and control (e.g., β-actin) primers and probes may be obtained commercially from, for example, Perkin Elmer/Applied Biosystems (Foster City, Calif.). To quantitate the amount of specific RNA in a sample, a standard curve is generated alongside using a plasmid containing the gene of interest. Standard curves may be generated using the Ct values determined in the real-time PCR, which are related to the initial cDNA concentration used in the assay. Standard dilutions ranging from 10-10⁶ copies of the gene of interest are generally sufficient. In addition, a standard curve is generated for the control sequence. This permits standardization of initial RNA content of a tissue sample to the amount of control for comparison purposes.

Polynucleotide variants may generally be prepared by any method known in the art, including chemical synthesis by, for example, solid phase phosphoramidite chemical synthesis. Modifications in a polynucleotide sequence may also be introduced using standard mutagenesis techniques, such as oligonucleotide-directed site-specific mutagenesis (see Adelman et al., DNA 2:183, 1983). Alternatively, RNA molecules may be generated by in vitro or in vivo transcription of DNA sequences encoding an ovarian carcinoma antigen, or portion thereof, provided that the DNA is incorporated into a vector with a suitable RNA polymerase promoter (such as T7 or SP6). Certain portions may be used to prepare an encoded polypeptide, as described herein. In addition, or alternatively, a portion may be administered to a patient such that the encoded polypeptide is generated in vivo.

A portion of a sequence complementary to a coding sequence (i.e., an antisense polynucleotide) may also be used as a probe or to modulate gene expression. cDNA constructs that can be transcribed into antisense RNA may also be introduced into cells or tissues to facilitate the production of antisense RNA. An antisense polynucleotide may be used, as described herein, to inhibit expression of an ovarian carcinoma protein. Antisense technology can be used to control gene expression through triple-helix formation, which compromises the ability of the double helix to open sufficiently for the binding of polymerases, transcription factors or regulatory molecules (see Gee et al., In Huber and Carr, Molecular and Immunologic Approaches, Futura Publishing Co. (Mt. Kisco, N.Y.; 1994). Alternatively, an antisense molecule may be designed to hybridize with a control region of a gene (e.g., promoter, enhancer or transcription initiation site), and block transcription of the gene; or to block translation by inhibiting binding of a transcript to ribosomes.

Any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

Nucleotide sequences as described herein may be joined to a variety of other nucleotide sequences using established recombinant DNA techniques. For example, a polynucleotide may be cloned into any of a variety of cloning vectors, including plasmids, phagemids, lambda phage derivatives and cosmids. Vectors of particular interest include expression vectors, replication vectors, probe generation vectors and sequencing vectors. In general, a vector will contain an origin of replication functional in at least one organism, convenient restriction endonuclease sites and one or more selectable markers. Other elements will depend upon the desired use, and will be apparent to those of ordinary skill in the art.

Within certain embodiments, polynucleotides may be formulated so as to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, or vaccinia or other pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

Other formulations for therapeutic purposes include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Ovarian Carcinoma Polypeptides

Within the context of the present invention, polypeptides may comprise at least an immunogenic portion of an ovarian carcinoma protein or a variant thereof, as described herein. Certain ovarian carcinoma proteins are ovarian carcinoma antigens that are expressed by ovarian tumor cells and react detectably within an immunoassay (such as an ELISA) with antisera generated against serum from an immunodeficient animal implanted with an ovarian tumor. Other ovarian carcinoma proteins are encoded by ovarian carcinoma polynucleotides recited herein. Polypeptides as described herein may be of any length. Additional sequences derived from the native protein and/or heterologous sequences may be present, and such sequences may (but need not) possess further immunogenic or antigenic properties.

An “immunogenic portion,” as used herein is a portion of an antigen that is recognized (i.e., specifically bound) by a B-cell and/or T-cell surface antigen receptor. Such immunogenic portions generally comprise at least 5 amino acid residues, more preferably at least 10, and still more preferably at least 20 amino acid residues of an ovarian carcinoma protein or a variant thereof. Preferred immunogenic portions are encoded by cDNA molecules isolated as described herein. Further immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with ovarian carcinoma protein-specific antibodies, antisera and/or T-cell lines or clones. As used herein, antisera and antibodies are “ovarian carcinoma protein-specific” if they specifically bind to an ovarian carcinoma protein (i.e., they react with the ovarian carcinoma protein in an ELISA or other immunoassay, and do not react detectably with unrelated proteins). Such antisera, antibodies and T cells may be prepared as described herein, and using well known techniques. An immunogenic portion of a native ovarian carcinoma protein is a portion that reacts with such antisera, antibodies and/or T-cells at a level that is not substantially less than the reactivity of the full length polypeptide (e.g., in an ELISA and/or T-cell reactivity assay). Such immunogenic portions may react within such assays at a level that is similar to or greater than the reactivity of the full length protein. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As noted above, a composition may comprise a variant of a native ovarian carcinoma protein. A polypeptide “variant,” as used herein, is a polypeptide that differs from a native ovarian carcinoma protein in one or more substitutions, deletions, additions and/or insertions, such that the immunogenicity of the polypeptide is not substantially diminished. In other words, the ability of a variant to react with ovarian carcinoma protein-specific antisera may be enhanced or unchanged, relative to the native ovarian carcinoma protein, or may be diminished by less than 50%, and preferably less than 20%, relative to the native ovarian carcinoma protein. Such variants may generally be identified by modifying one of the above polypeptide sequences and evaluating the reactivity of the modified polypeptide with ovarian carcinoma protein-specific antibodies or antisera as described herein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants preferably exhibit at least about 70%, more preferably at least about 90% and most preferably at least about 95% identity to the native polypeptide. Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides may be prepared using any of a variety of well known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having fewer than about 100 amino acids, and generally fewer than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Applied BioSystems, Inc. (Foster City, Calif.), and may be operated according to the manufacturer's instructions.

Within certain specific embodiments, a polypeptide may be a fusion protein that comprises multiple polypeptides as described herein, or that comprises one polypeptide as described herein and a known tumor antigen, such as an ovarian carcinoma protein or a variant of such a protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Fusion proteins are also provided that comprise a polypeptide of the present invention together with an unrelated immunogenic protein. Preferably the immunogenic protein is capable of eliciting a recall response. Examples of such proteins include tetanus, tuberculosis and hepatitis proteins (see, for example, Stoute et al. New Engl. J. Med., 336:86-91, 1997).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen present cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Binding Agents

The present invention further provides agents, such as antibodies and antigen-binding fragments thereof, that specifically bind to an ovarian carcinoma protein. As used herein, an antibody, or antigen-binding fragment thereof, is said to “specifically bind” to an ovarian carcinoma protein if it reacts at a detectable level (within, for example, an ELISA) with an ovarian carcinoma protein, and does not react detectably with unrelated proteins under similar conditions. As used herein, “binding” refers to a noncovalent association between two separate molecules such that a “complex” is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant maybe determined using methods well known in the art.

Binding agents may be further capable of differentiating between patients with and without a cancer, such as ovarian cancer, using the representative assays provided herein. In other words, antibodies or other binding agents that bind to a ovarian carcinoma antigen will generate a signal indicating the presence of a cancer in at least about 20% of patients with the disease, and will generate a negative signal indicating the absence of the disease in at least about 90% of individuals without the cancer. To determine whether a binding agent satisfies this requirement, biological samples (e.g., blood, sera, leukophoresis, urine and/or tumor biopsies) from patients with and without a cancer (as determined using standard clinical tests) may be assayed as described herein for the presence of polypeptides that bind to the binding agent. It will be apparent that a statistically significant number of samples with and without the disease should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

Also provided herein are anti-idiotypic antibodies that mimic an immunogenic portion of an ovarian carcinoma protein. Such antibodies may be raised against an antibody, or antigen-binding fragment thereof, that specifically binds to an immunogenic portion of an ovarian carcinoma protein, using well known techniques. Anti-idiotypic antibodies that mimic an immunogenic portion of an ovarian carcinoma protein are those antibodies that bind to an antibody, or antigen-binding fragment thereof, that specifically binds to an immunogenic portion of an ovarian carcinoma protein, as described herein.

T Cells

Immunotherapeutic compositions may also, or alternatively, comprise T cells specific for an ovarian carcinoma protein. Such cells may generally be prepared in vitro or ex vivo, using standard procedures. For example, T cells may be present within (or isolated from) bone marrow, peripheral blood or a fraction of bone marrow or peripheral blood of a mammal, such as a patient, using a commercially available cell separation system, such as the CEPRATE™ system, available from CellPro Inc., Bothell Wash. (see also U.S. Pat. No. 5,240,856; U.S. Pat. No. 5,215,926; WO 89/06280; WO 91/16116 and WO 92/07243). Alternatively, T cells may be derived from related or unrelated humans, non-human animals, cell lines or cultures.

T cells may be stimulated with an ovarian carcinoma polypeptide, polynucleotide encoding an ovarian carcinoma polypeptide and/or an antigen presenting cell (APC) that expresses such a polypeptide. Such stimulation is performed under conditions and for a time sufficient to permit the generation of T cells that are specific for the polypeptide. Preferably, an ovarian carcinoma polypeptide or polynucleotide is present within a delivery vehicle, such as a microsphere, to facilitate the generation of specific T cells.

T cells are considered to be specific for an ovarian carcinoma polypeptide if the T cells kill target cells coated with an ovarian carcinoma polypeptide or expressing a gene encoding such a polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with an ovarian carcinoma polypeptide (200 ng/ml-100 μg/ml, preferably 100 ng/ml-25 μg/ml) for 3-7 days should result in at least a two fold increase in proliferation of the T cells and/or contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998). T cells that have been activated in response to an ovarian carcinoma polypeptide, polynucleotide or ovarian carcinoma polypeptide-expressing APC may be CD4⁺ and/or CD8⁺. Ovarian carcinoma polypeptide-specific T cells may be expanded using standard techniques. Within preferred embodiments, the T cells are derived from a patient or a related or unrelated donor and are administered to the patient following stimulation and expansion.

For therapeutic purposes, CD4⁺ or CD8⁺ T cells that proliferate in response to an ovarian carcinoma polypeptide, polynucleotide or APC can be expanded in number either in vitro or in vivo. Proliferation of such T cells in vitro may be accomplished in a variety of ways. For example, the T cells can be re-exposed to an ovarian carcinoma polypeptide, with or without the addition of T cell growth factors, such as interleukin-2, and/or stimulator cells that synthesize an ovarian carcinoma polypeptide. Alternatively, one or more T cells that proliferate in the presence of an ovarian carcinoma polypeptide can be expanded in number by cloning. Methods for cloning cells are well known in the art, and include limiting dilution. Following expansion, the cells may be administered back to the patient as described, for example, by Chang et al., Crit. Rev. Oncol. Hematol. 22:213, 1996.

Pharmaceutical Compositions and Vaccines

Within certain aspects, polypeptides, polynucleotides, binding agents and/or immune system cells as described herein may be incorporated into pharmaceutical compositions or vaccines. Pharmaceutical compositions comprise one or more such compounds or cells and a physiologically acceptable carrier. Vaccines may comprise one or more such compounds or cells and a non-specific immune response enhancer. A non-specific immune response enhancer may be any substance that enhances an immune response to an exogenous antigen. Examples of non-specific immune response enhancers include adjuvants, biodegradable microspheres (e.g., polylactic galactide) and liposomes (into which the compound is incorporated; see e.g., Fullerton, U.S. Pat. No. 4,235,877). Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound within the composition or vaccine.

A pharmaceutical composition or vaccine may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., PNAS 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., PNAS 91:215-219, 1994; Kass-Eisler et al., PNAS 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes using well known technology.

Any of a variety of non-specific immune response enhancers may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.), Merck Adjuvant 65 (Merck and Company, Inc., Rahway, N.J.), alum, biodegradable microspheres, monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, IL-5, IL-6, IL-10 and TNF-β) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Ribi ImmunoChem Research Inc. (Hamilton, M T; see U.S. Pat. Nos. 4,436,727; 4,877,611; 4,866,034 and 4,912,094). Also preferred is AS-2 (SmithKline Beecham). CpG-containing oligonucleotides (in which the CpG dinucleotide is unmethylated) also induce a predominantly Thl response. Such oligonucleotides are well known and are described, for example, in WO 96/02555. Another preferred adjuvant is a saponin, preferably QS21, which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprises an oil-in-water emulsion and tocopherol. A particularly potent adjuvant formulation involving QS21, 3D-MPL and tocopherol in an oil-in-water emulsion is described in WO 95/17210. Any vaccine provided herein may be prepared using well known methods that result in a combination of antigen, immune response enhancer and a suitable carrier or excipient.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro) and based on the lack of differentiation markers of B cells (CD1 9 and CD20), T cells (CD3), monocytes (CD14) and natural killer cells (CD56), as determined using standard assays. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor, mannose receptor and DEC-205 marker. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80 and CD86).

APCs may generally be transfected with a polynucleotide encoding a ovarian carcinoma antigen (or portion or other variant thereof) such that the antigen, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Cancer Therapy

In further aspects of the present invention, the compositions described herein may be used for immunotherapy of cancer, such as ovarian cancer. Within such methods, pharmaceutical compositions and vaccines are typically administered to a patient. As used herein, a “patient” refers to any warm-blooded animal, preferably a human. A patient may or may not be afflicted with cancer. Accordingly, the above pharmaceutical compositions and vaccines may be used to prevent the development of a cancer or to treat a patient afflicted with a cancer. Within certain preferred embodiments, a patient is afflicted with ovarian cancer. Such cancer may be diagnosed using criteria generally accepted in the art, including the presence of a malignant tumor. Pharmaceutical compositions and vaccines may be administered either prior to or following surgical removal of primary tumors and/or treatment such as administration of radiotherapy or conventional chemotherapeutic drugs.

Within certain embodiments, immunotherapy may be active immunotherapy, in which treatment relies on the in vivo stimulation of the endogenous host immune system to react against tumors with the administration of immuno response-modifying agents (such as tumor vaccines, bacterial adjuvants and/or cytokines).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T lymphocytes (such as CD8⁺ cytotoxic T lymphocytes and CD⁴⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into stem cells taken from a patient and clonally propagated in vitro for autologous transplant back into the same patient.

Routes and frequency of administration, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration), orally or in the bed of a resected tumor. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 100 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to an ovarian carcinoma antigen generally correlate with an improved clinical outcome. Such immune responses may generally be evaluated using standard proliferation, cytotoxicity or cytokine assays, which may be performed using samples obtained from a patient before and after treatment.

Methods for Detecting Cancer

In general, a cancer may be detected in a patient based on the presence of one or more ovarian carcinoma proteins and/or polynucleotides encoding such proteins in a biological sample (such as blood, sera, urine and/or tumor biopsies) obtained from the patient. In other words, such proteins may be used as markers to indicate the presence or absence of a cancer such as ovarian cancer. In addition, such proteins may be useful for the detection of other cancers. The binding agents provided herein generally permit detection of the level of protein that binds to the agent in the biological sample. Polynucleotide primers and probes may be used to detect the level of mRNA encoding a tumor protein, which is also indicative of the presence or absence of a cancer. In general, an ovarian carcinoma-associated sequence should be present at a level that is at least three fold higher in tumor tissue than in normal tissue.

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length ovarian carcinoma proteins and portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with ovarian cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20™. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as ovarian cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use ovarian carcinoma polypeptides to detect antibodies that bind to such polypeptides in a biological sample. The detection of such ovarian carcinoma protein specific antibodies may correlate with the presence of a cancer.

A cancer may also, or alternatively, be detected based on the presence of T cells that specifically react with an ovarian carcinoma protein in a biological sample. Within certain methods, a biological sample comprising CD4⁺ and/or CD8⁺ T cells isolated from a patient is incubated with an ovarian carcinoma protein, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with an ovarian carcinoma protein (e.g., 5-25 μg/ml). It may be desirable to incubate another aliquot of a T cell sample in the absence of ovarian carcinoma protein to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding an ovarian carcinoma protein in a biological sample. For example, at least two oligonucleotide primers may be employed in a polymerase chain reaction (PCR) based assay to amplify a portion of an ovarian carcinoma protein cDNA derived from a biological sample, wherein at least one of the oligonucleotide primers is specific for (i.e., hybridizes to) a polynucleotide encoding the ovarian carcinoma protein. The amplified cDNA is then separated and detected using techniques well known in the art, such as gel electrophoresis. Similarly, oligonucleotide probes that specifically hybridize to a polynucleotide encoding an ovarian carcinoma protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding an ovarian carcinoma protein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence provided herein. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample such as a biopsy tissue and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another embodiment, ovarian carcinoma proteins and polynucleotides encoding such proteins may be used as markers for monitoring the progression of cancer. In this embodiment, assays as described above for the diagnosis of a cancer may be performed over time, and the change in the level of reactive polypeptide(s) evaluated. For example, the assays may be performed every 24-72 hours for a period of 6 months to 1 year, and thereafter performed as needed. In general, a cancer is progressing in those patients in whom the level of polypeptide detected by the binding agent increases over time. In contrast, the cancer is not progressing when the level of reactive polypeptide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple ovarian carcinoma protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to an ovarian carcinoma protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding an ovarian carcinoma protein in a biological sample. Such kits generally comprise at least one oligonucleotide probe or primer, as described above, that hybridizes to a polynucleotide encoding an ovarian carcinoma protein. Such an oligonucleotide may be used, for example, within a PCR or hybridization assay. Additional components that may be present within such kits include a second oligonucleotide and/or a diagnostic reagent or container to facilitate the detection of a polynucleotide encoding an ovarian carcinoma protein.

The following Examples are offered by way of illustration and not by way of limitation.

### EXAMPLES

### Example 1

### Identification of Ovarian Tumor Antigen cDNAs

This Example illustrates the identification of cDNA molecules encoding ovarian tumor antigens.

Patient sera (from two human patients with ovarian cancer) was adsorbed against E. coli and used at a 1:200 dilution in a serological expression screen performed as described in Sambrook et al., Molecular Cloning: A Laboratory Manual, Cold Spring Harbor Laboratories, Cold Spring Harbor, N.Y., 1989. The library screened was made from a SCID-derived human ovarian tumor (OV9334) using a directional RH oligo(dT) priming cDNA library construction kit and the λScreen vector (Novagen). Approximately 600,000 pfu of the amplified OV9334 library were screened.

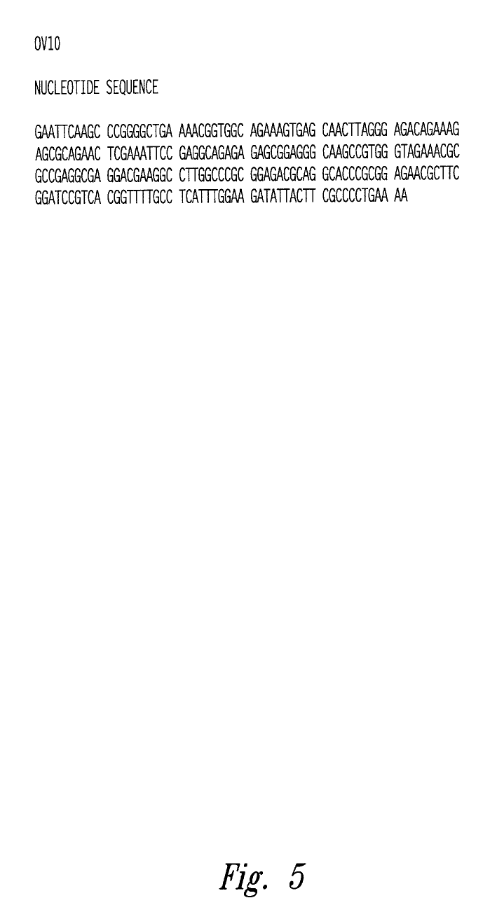

In a first screen, three novel clones were identified (OV10, OV17 and OV41), and the partial sequences are provided in FIGS. 5, 8 and 13 (SEQ ID NO: 5, 8 and 13), respectively. Additional sequences matched previously identified genes, which were not previously known to be associated with ovarian carcinoma. These sequences are identified in Table I.

TABLE I
Additional Ovarian Carcinoma Antigen Partial Sequences

| Sequence | Match |
|---|---|
| OV6 (SEQ ID NO: 3) | High Mobility Group Protein 14 |
| OV2 (SEQ ID NO: 1) | Hypothetical 33.4 kD Protein |
| OV3 (SEQ ID NO: 2) | High Mobility Group Protein 17 |
| OV9 (SEQ ID NO: 4) | Integrin Alpha-3 |
| OV12 (SEQ ID NO: 6) | Filamental Protein |
| OV14 (SEQ ID NO: 7) | Ubiquitin-Conjugating Enzyme |
| OV18 (SEQ ID NO: 9) | Alpha-Glucosidase I |
| OV23 (SEQ ID NO: 10) | Ribosomal Protein L10 |
| OV24 (SEQ ID NO: 11) | Mitochondrial Gene |
| OV27 (SEQ ID NO: 12) | Ribonucleoprotein |
| OV54 (SEQ ID NO: 14) | Ribosomal Protein L18 |
| OV57 (SEQ ID NO: 15) | Ribosomal Protein S10 |

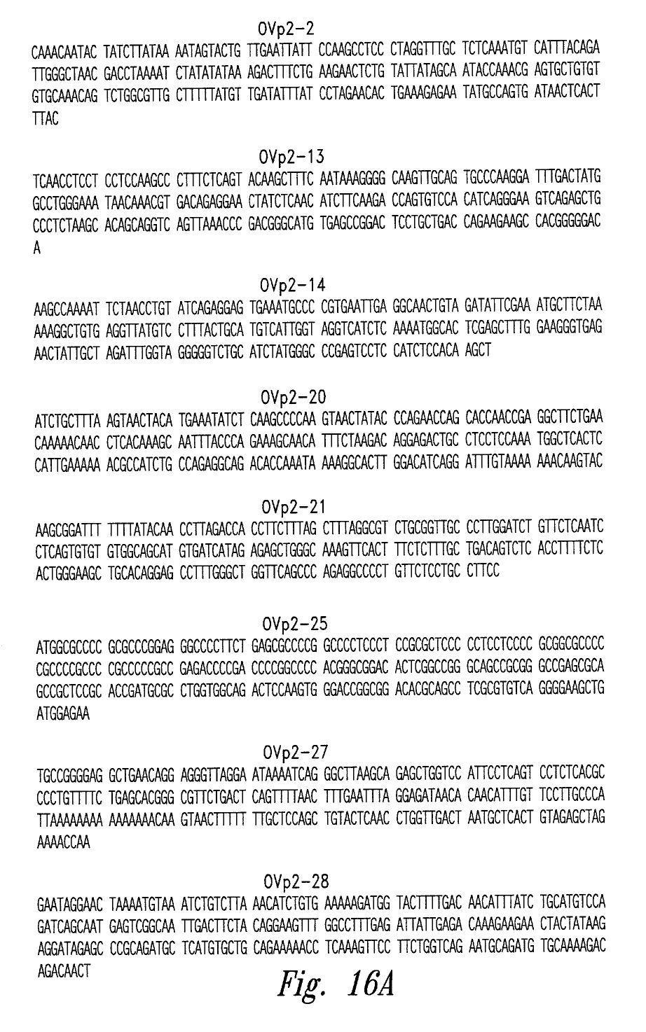

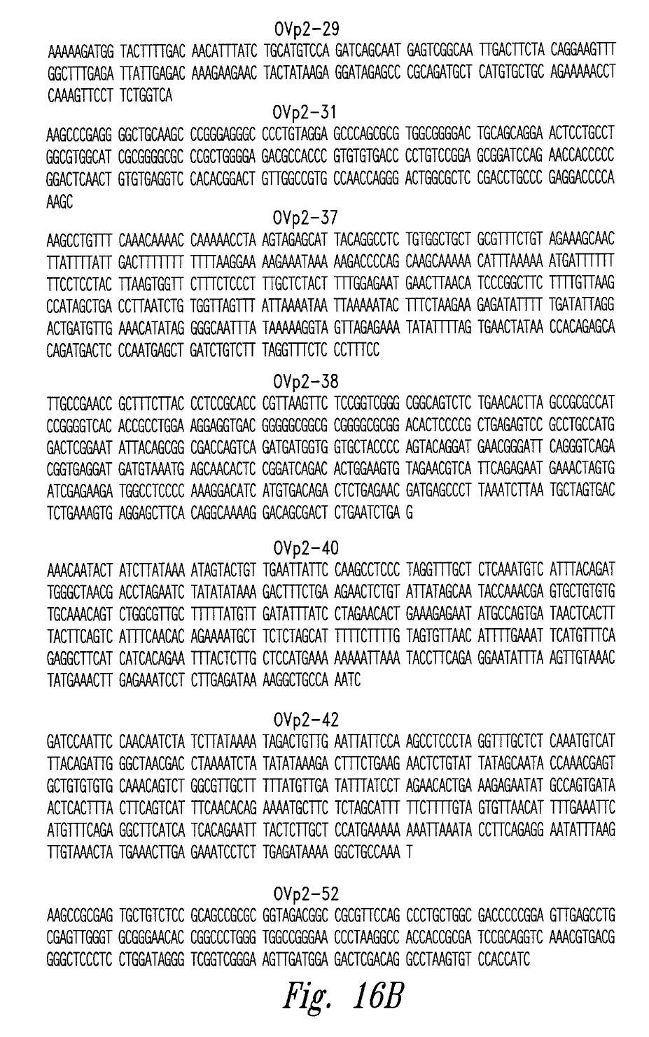

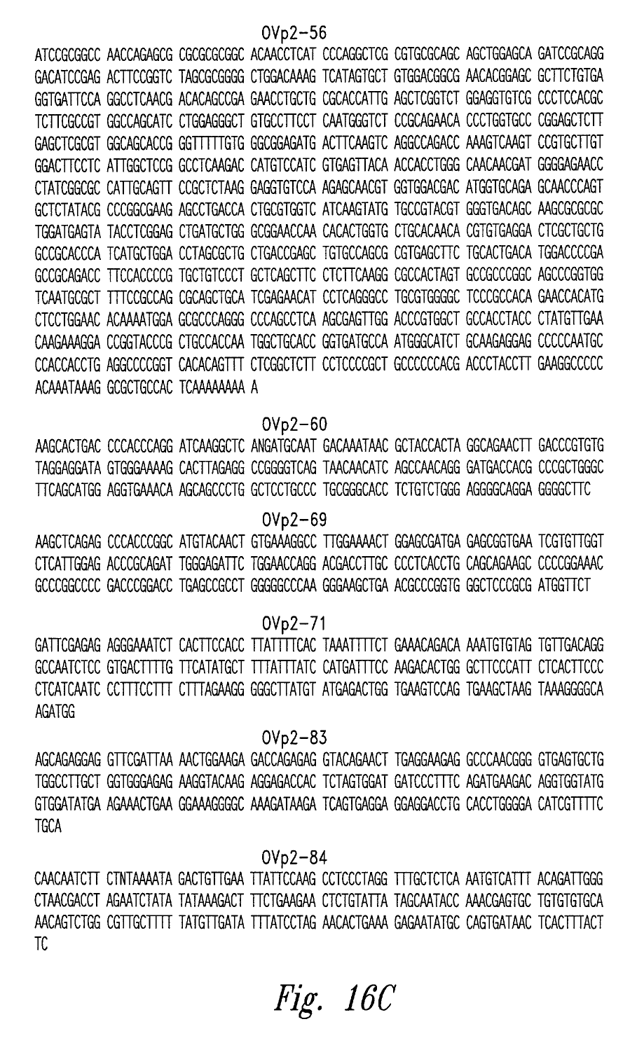

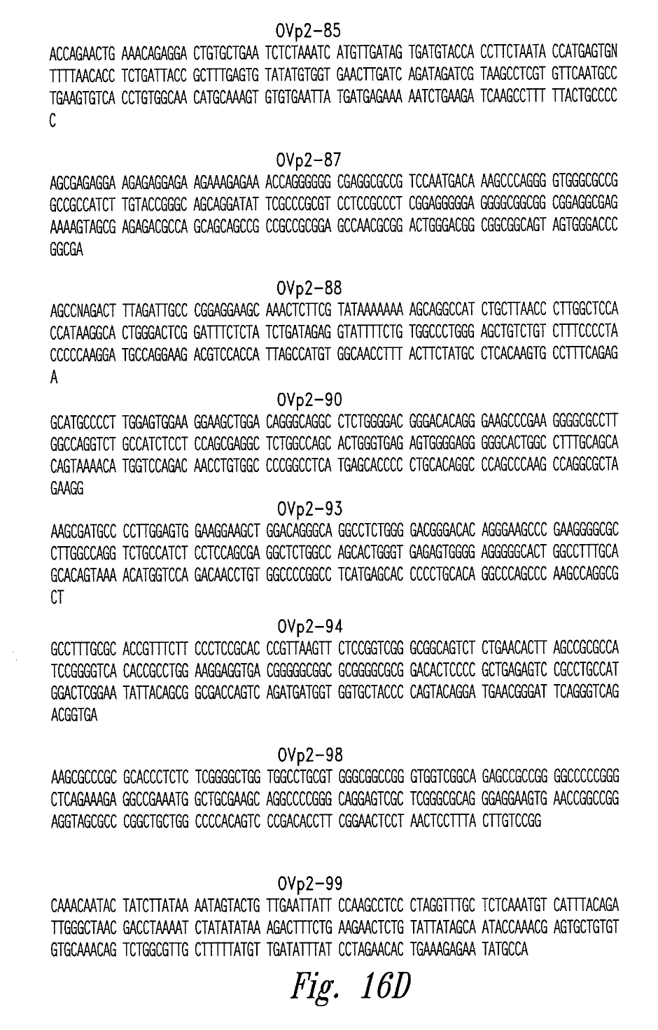

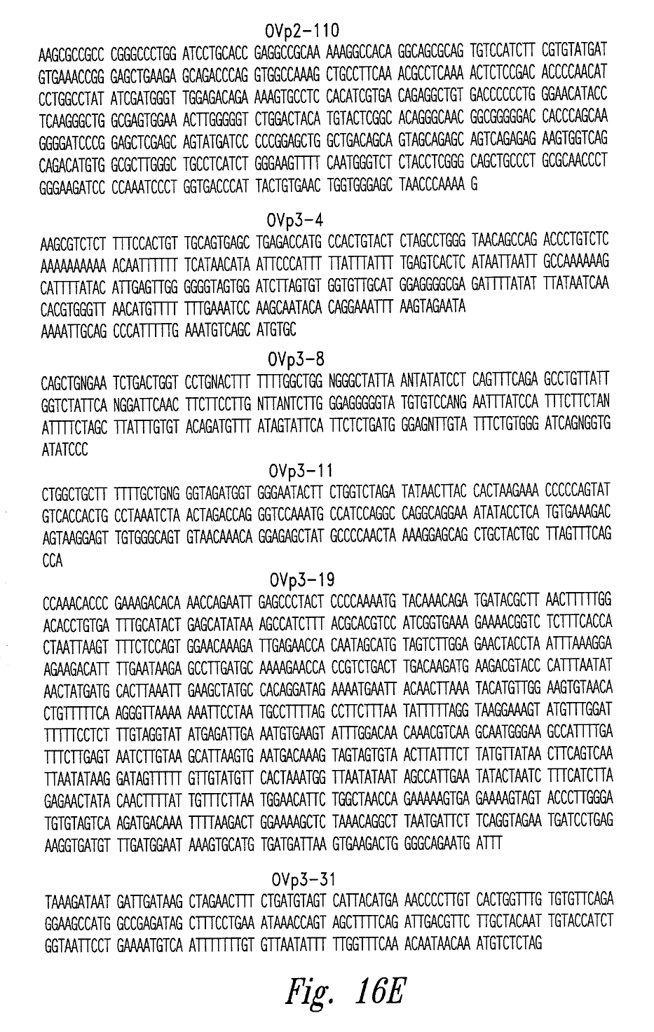

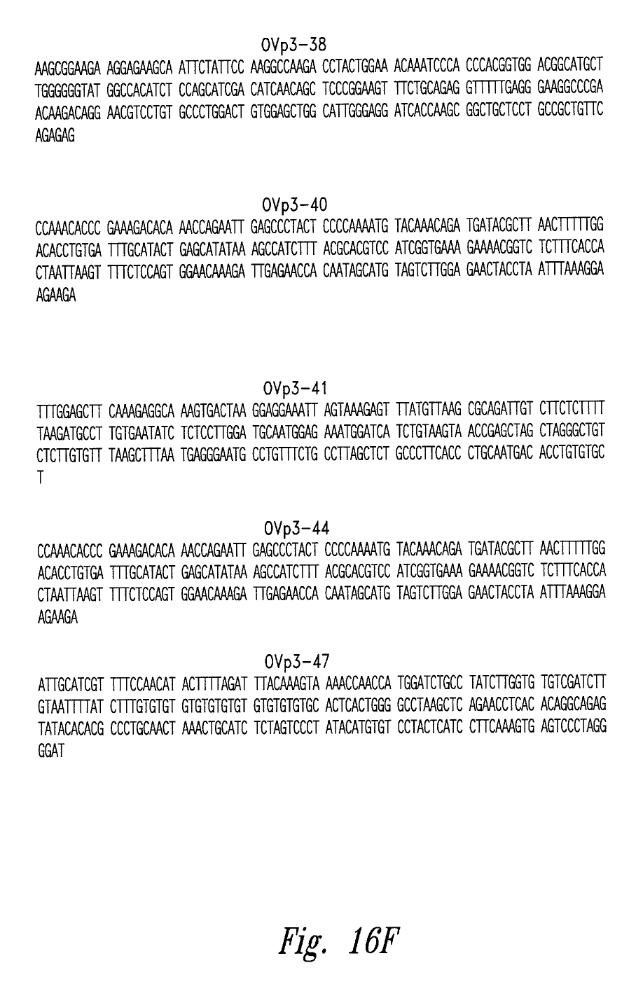

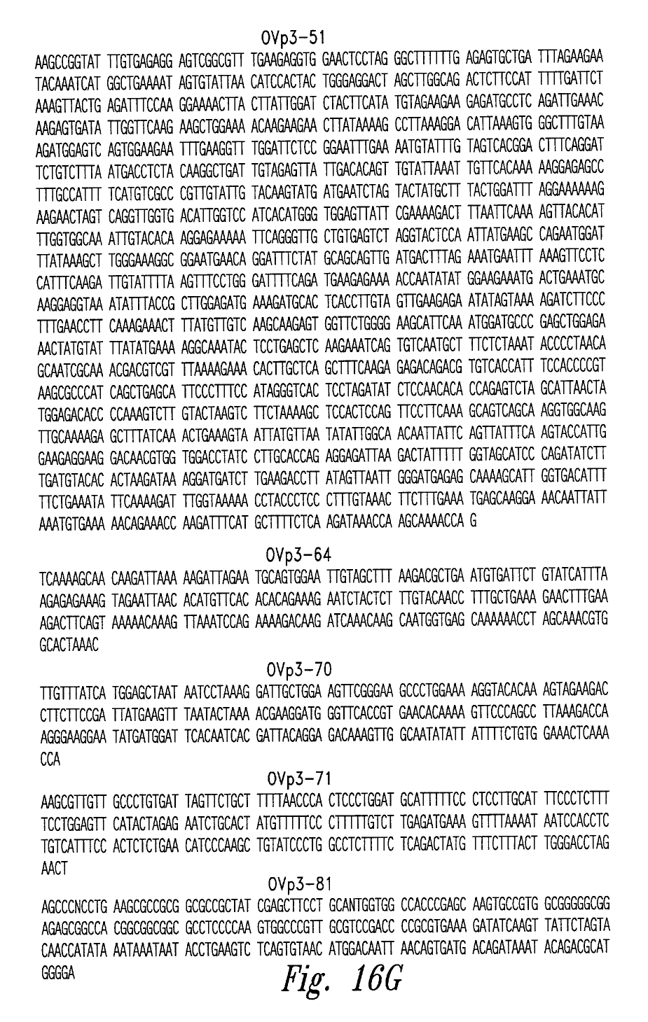

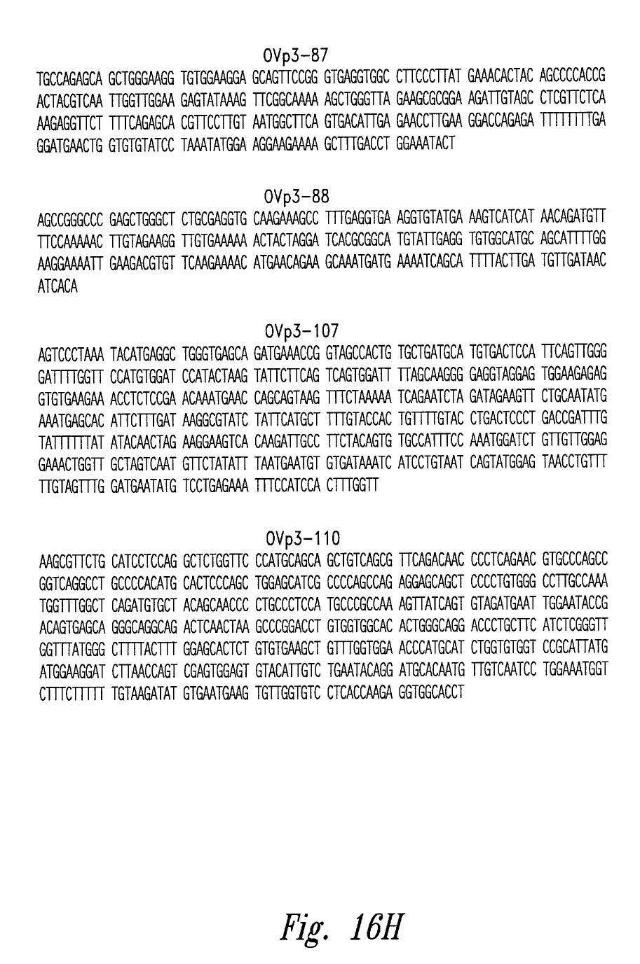

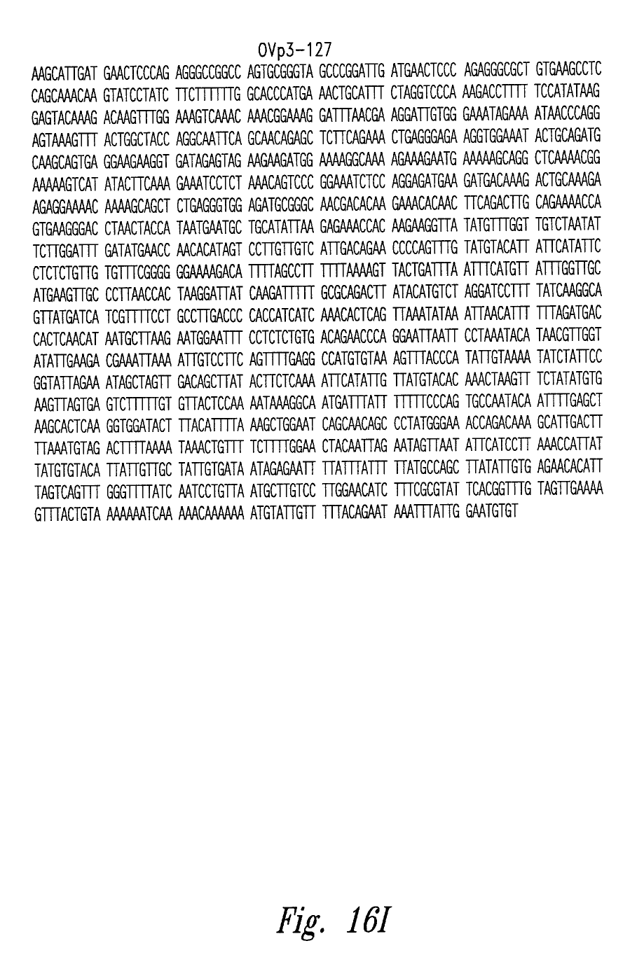

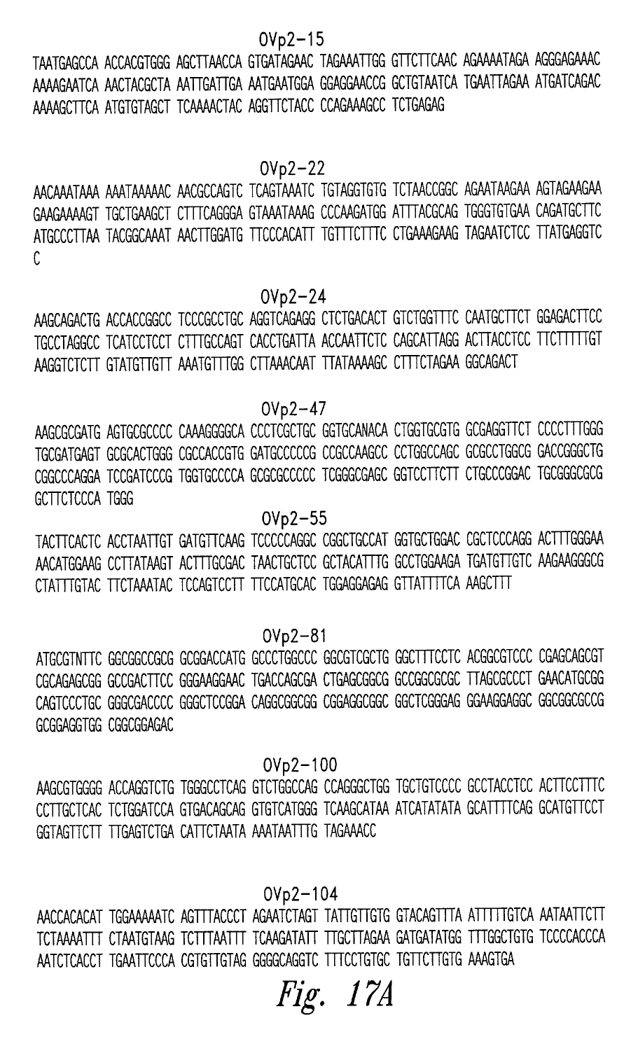

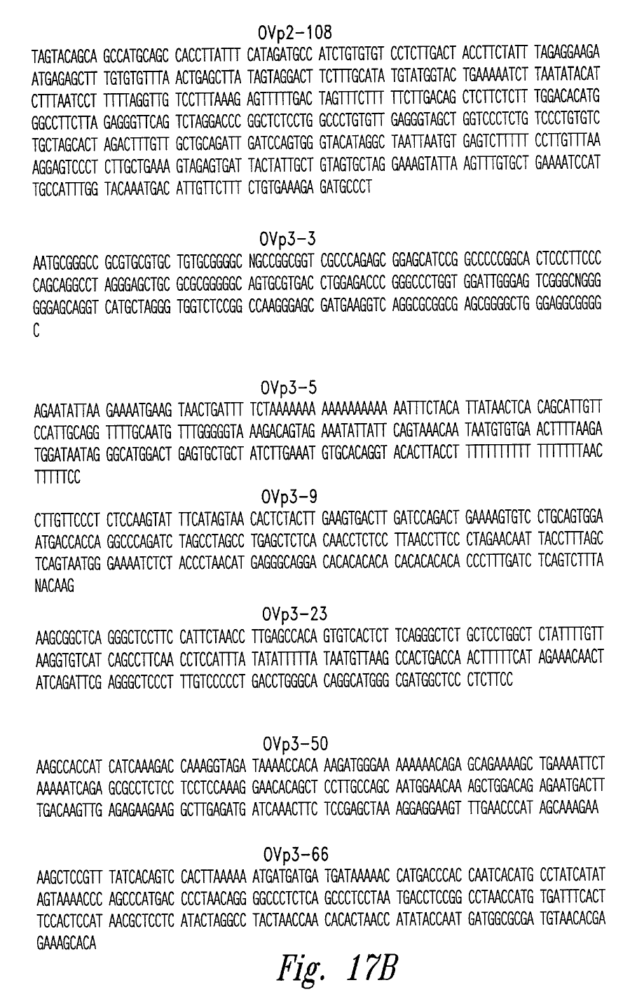

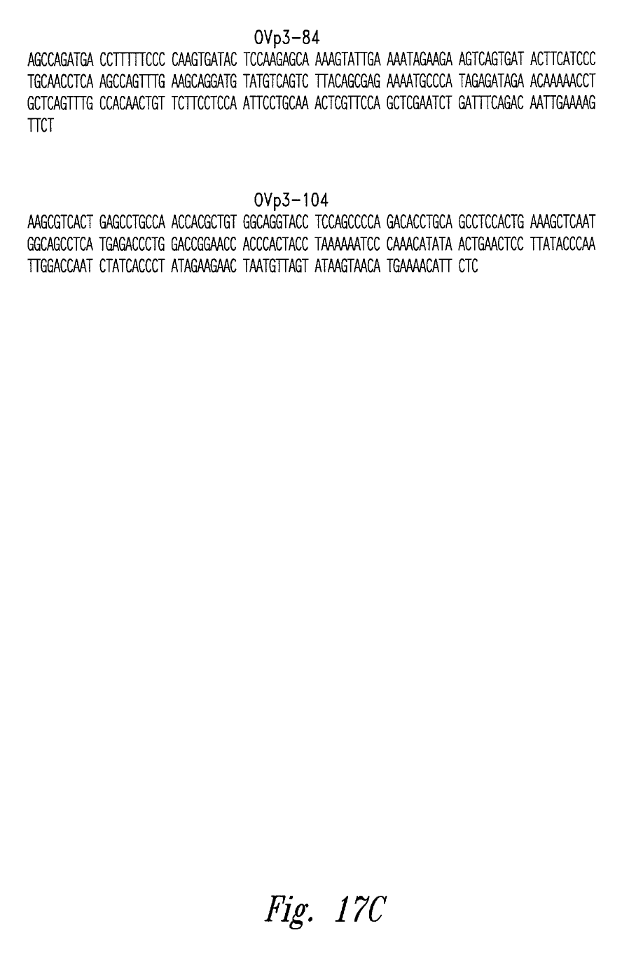

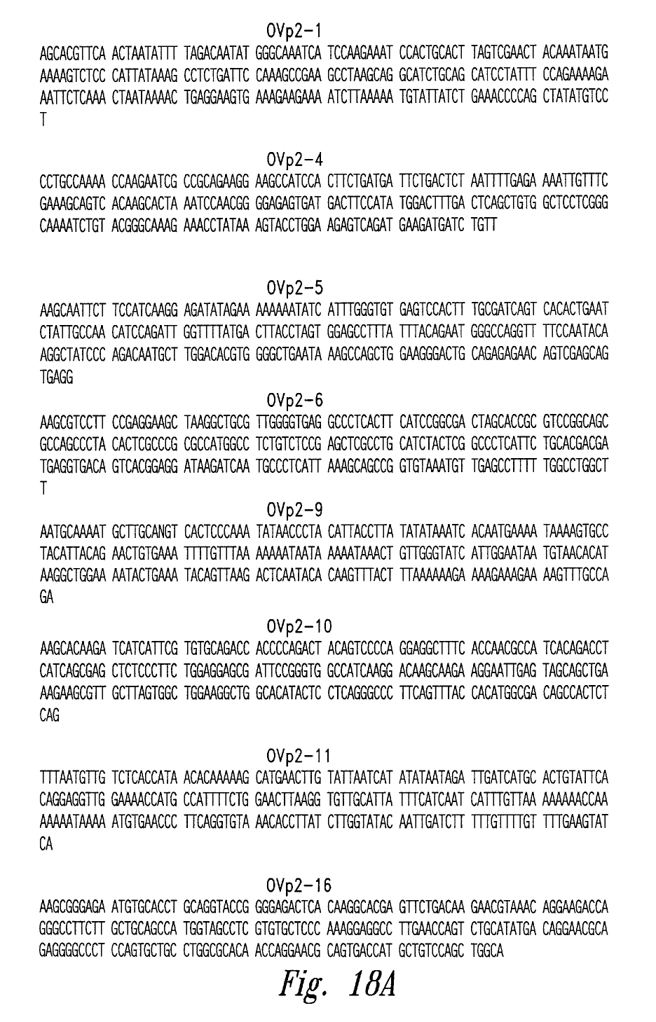

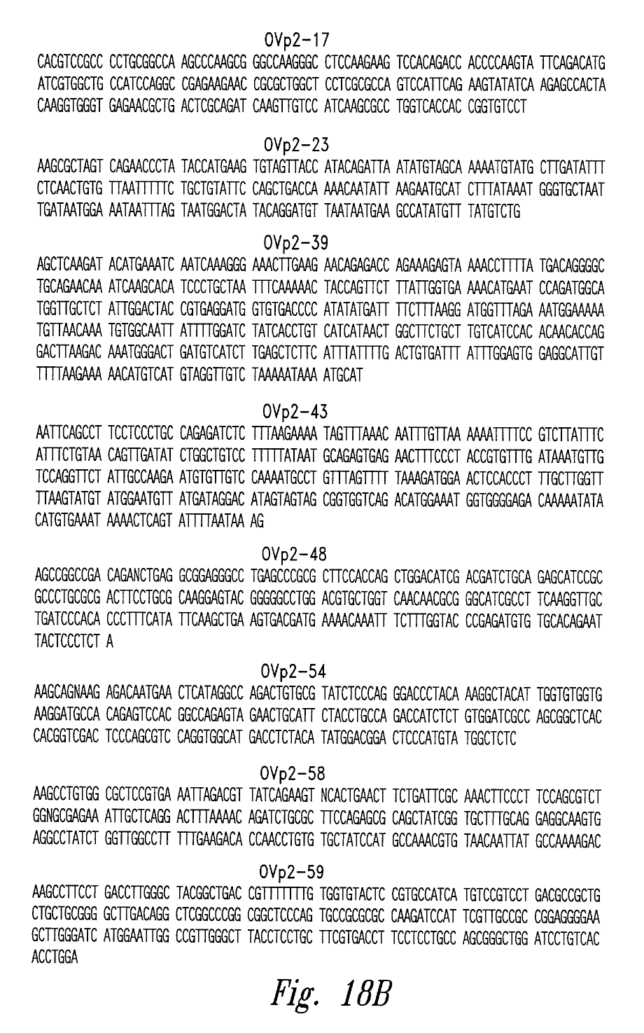

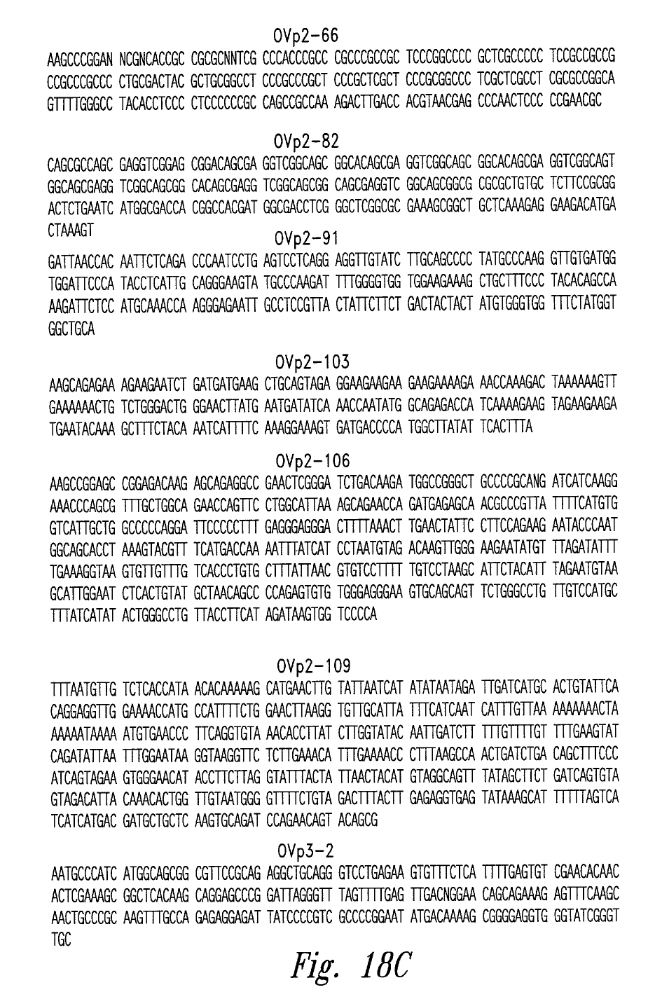

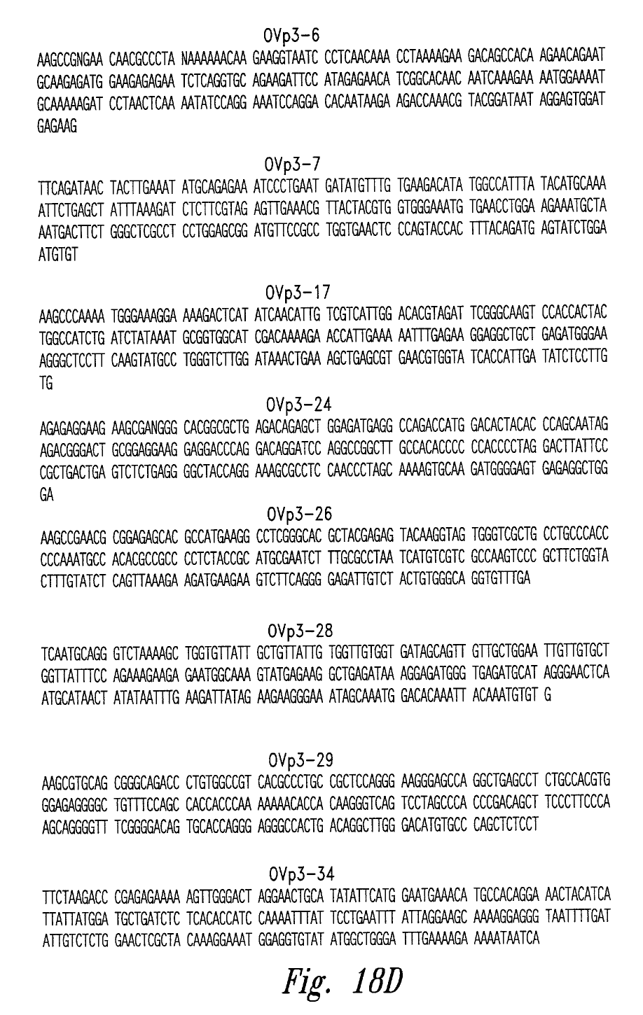

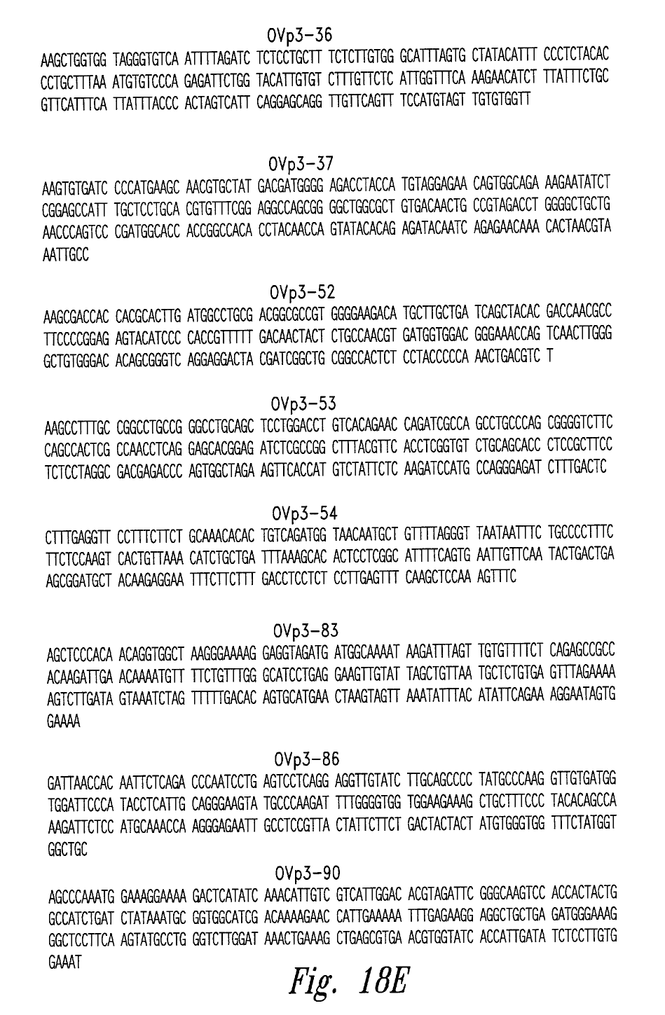

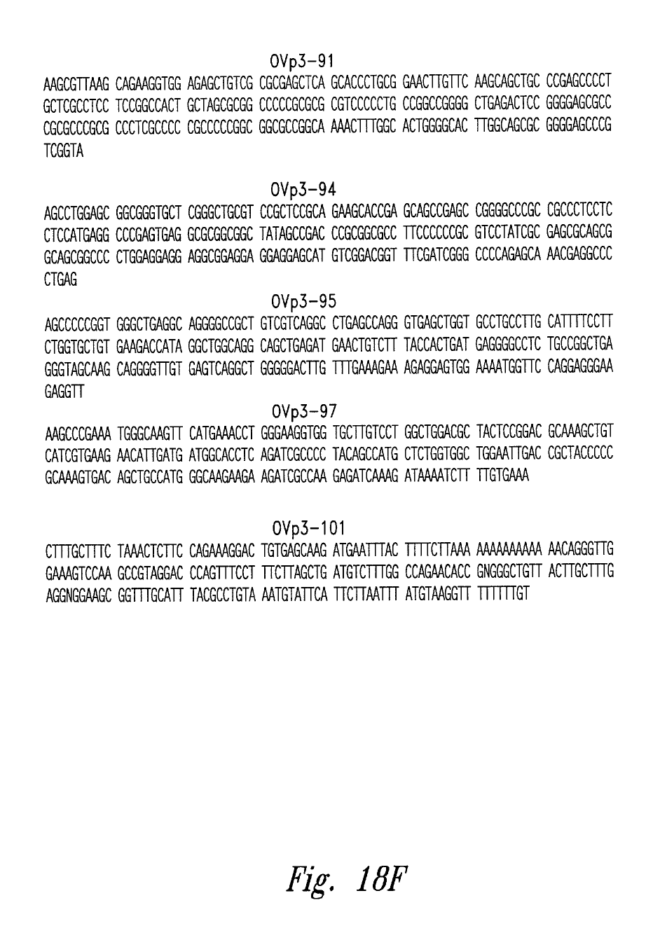

Within a second screen, further clones were identified. Homologies for these clones are provided in Table II. Sequences of novel clones are provided in FIGS. 16A-16I (SEQ ID NO:19-68). Those clones for which homology was found in the DNA sequence, but not the protein sequence, are provided in FIGS. 17A-17C (SEQ ID NO:69-85), and those that matched previously identified DNA and protein sequences, which were not previously known to be associated with ovarian carcinoma, are provided in FIGS. 18A-18F (SEQ ID NO:86-129).

TABLE II
Further Ovarian Carcinoma Antigen Partial Sequences

| | SEQ | | |
|---|---|---|---|
| Sequence | ID | DNA Homology | Protein Homology |
| OVp2-1 | 86 | AND - 1 | AND - 1 |
| OVp2-2 | 19 | Novel | Novel |
| OVp2-4 | 87 | Topoisom. II | Topoisom. II |
| OVp2-5 | 88 | cAMP phosphodiesterase | cAMP phosphodiesterase |
| OVp2-6 | 89 | P1 | P1 |
| OVp2-9 | 90 | kinase PRKARIA | kinase PRKARIA |
| OVp2-10 | 91 | Human RNA polym. II | Human RNA polym. II |
| OVp2-11 | 92 | Mu. p162 | Mu. p162 |
| OVp2-13 | 20 | Novel | Novel |
| OVp2-14 | 21 | Novel | Novel |
| OVp2-15 | 69 | Hu. BAC G5541B18 | Novel |
| OVp2-16 | 93 | Hu. fanconi anemia | FAA |
| | | mRNA | |
| OVp2-17 | 94 | Hu. histone H1 | Histone H1 |
| OVp2-20 | 22 | Novel | Novel |
| OVp2-21 | 23 | Novel | Novel |
| OVp2-22 | 70 | Hu KIAA0642 mRNA | Novel |
| OVp2-23 | 95 | Hu. lysophospholipase | Hu. lysophospholipase |
| | | mRNA | mRNA |
| OVp2-24 | 71 | Hu. Chrom 16 cosmid | Novel |
| | | clone 400D1 | |
| OVp2-25 | 24 | Novel | Novel; similar to PHD |
| | | | finger protein |
| OVp2-27 | 25 | Novel | Novel |
| OVp2-28 | 26 | Novel | Novel |
| OVp2-29 | 27 | Novel | Novel |
| OVp2-31 | 28 | Novel | Novel |
| OVp2-37 | 29 | Novel | Novel |
| OVp2-38 | 30 | Novel | Novel |
| OVp2-39 | 96 | Human Tumor protein | Human Tumor protein |
| OVp2-40 | 31 | Novel | Novel |
| OVp2-42 | 32 | Novel | Novel |
| OVp2-43 | 97 | nucleophosmin | nucleophosmin |
| OVp2-47 | 72 | Hu PAC DJ0751H13 | Novel; zinc finger-like |
| OVp2-48 | 98 | Human Carbonyl | Carbonyl Reductase |
| | | Reductase | |
| OVp2-52 | 33 | Novel | Novel |
| OVp2-54 | 99 | Mu., Hu. chromatin | Mu., Hu. chromatin |
| | | structrl pro. | structrl pro. |
| OVp2-55 | 73 | Hu. mRNA clone A33187 | Novel; similar to laminin |
| OVp2-56 | 34 | Novel | Novel |
| OVp2-58 | 100 | H3.3 Histone | H3.3 Histone |
| OVp2-59 | 101 | cyt.C oxidase subunit | cyt.C oxidase subunit |
| OVp2-60 | 35 | Novel | Novel |
| OVp2-66 | 102 | alpha-CP1; rnp-assoc. | alpha-CP1; rnp-assoc. |
| OVp2-69 | 36 | Novel | Novel |
| OVp2-71 | 37 | Novel | Novel |
| OVp2-81 | 74 | HuCpG DNA clone 11B11 | Novel |
| OVp2-82 | 103 | Human KIAA0111 gene | Euk Init'n Factor 4A-like |
| OVp2-83 | 38 | Novel | Novel |
| OVp2-84 | 39 | Novel | Novel |
| OVp2-85 | 40 | Novel | Novel |
| OVp2-87 | 41 | Novel | Novel |
| OVp2-88 | 42 | Novel | Novel |
| OVp2-90 | 43 | Novel | Novel |
| OVp2-91 | 104 | Mu, Hu cathepsinC | Mu, Hu cathepsinC |
| OVp2-93 | 44 | Novel | Novel |
| OVp2-94 | 45 | Novel | Novel |
| OVp2-98 | 46 | Novel; Carbonyl Reductase | Novel |
| | | (pig) | |
| OVp2-99 | 47 | Novel | Novel |
| OVp2-100 | 75 | Human KIAA0788 | Novel |
| OVp2-103 | 105 | Human tra gp96 | tra gp96 |
| OVp2-104 | 76 | Hu BAC clone 78c6 | Novel |
| OVp2-106 | 106 | Ubiq-conj. enz; carcinoma | Ubiquitin-conjugating |
| | | | enzyme |
| OVp2-108 | 77 | KIAA0249 | Novel |
| OVp2-109 | 107 | Mu. p162 | Mu. p162 |
| OVp2-110 | 48 | Novel | Novel |
| OVp3-2 | 108 | Mu. Acyl-CoA dehydrog. | Mu. Acyl-CoA |
| | | | dehydrog. |
| OVp3-3 | 78 | Hu. Chrom 17, hRPC.117 | Novel |
| OVp3-4 | 49 | Novel | Novel |
| OVp3-5 | 79 | Unkn. Hu. DNA; | Novel |
| OVp3-6 | 109 | Mu. retrotranspsn. | Mu. ORF; carcinoma |
| OVp3-7 | 110 | Hu. glypican4 | Glypican precursor |
| OVp3-8 | 50 | Novel | Novel |
| OVp3-9 | 80 | Mu. entactin | Novel |
| OVp3-11 | 51 | Novel | Novel |
| OVp3-17 | 111 | Elongation Factor 1 alpha | EF-1 alpha |
| OVp3-19 | 52 | Novel | Novel |
| OVp3-23 | 81 | Hu. RES4-4 (brain) | Novel |
| OVp3-24 | 112 | Hu. P8; mitogen | Hu. P8 |
| OVp3-26 | 113 | Hu ribosomal. L18 | Hu ribosomal. L18 |
| OVp3-28 | 114 | Hu KS/14 antigen | Tumor-assoc. |
| | | | glycoprotein |
| OVp3-29 | 115 | Hu. Actin-binding | Hu. Actin-binding |
| OVp3-31 | 53 | Novel | Novel |
| OVp3-34 | 116 | Hu DPM1 | Hu DPM1 |
| OVp3-36 | 117 | Hu PAC DJ269O05 | Hu. Line-1 homolog; |
| | | | putative p150 |
| OVp3-37 | 118 | Mu. fibronectin/precursor | fibronectin |
| | | (Hu) | |
| OVp3-38 | 54 | Novel | Novel |
| OVp3-40 | 55 | Novel | Novel |
| OVp3-41 | 56 | Novel | Novel |
| OVp3-44 | 57 | Novel | Novel |
| OVp3-47 | 58 | Novel | Novel |
| OVp3-50 | 82 | Chrom.; PAC | Novel; similar to p40, |
| | | | 40kD |
| OVp3-51 | 59 | Novel; 86% Mu. oncogene | Novel; Mu ect2 |
| | | ect2 | |
| OVp3-52 | 119 | RAC3 (Hu Rac3) | Ras-like; G protein; Rac3 |
| OVp3-53 | 120 | (Hu) alpha-enolase | alpha-enolase; carbonate |
| | | | hydratase(Hu) |
| OVp3-54 | 121 | Hu. Nuclr. NP220 | NP220 |
| OVp3-64 | 60 | Novel | Novel |
| OVp3-66 | 83 | Hu. mitochond. DNA | Novel; similar to cyt.C |
| | | | oxidase subunit |
| OVp3-70 | 61 | Novel | Novel |
| OVp3-71 | 62 | Novel | Novel |
| OVp3-81 | 63 | Novel | Novel |
| OVp3-83 | 122 | RNA binding factor | Unknown |
| OVp3-84 | 84 | Hu PAC RPCI3-197B17 | Novel |
| OVp3-86 | 123 | Cathepsin C | Cathepsin C |
| OVp3-87 | 64 | Novel | Novel |
| OVp3-88 | 65 | Novel | Novel |
| OVp3-90 | 124 | Human EF-1 alpha | Human EF-1 alpha |
| OVp3-91 | 125 | Human nuclear factor | similar to Hu. nuclear |
| | | NF-IL6 | factor NF-IL6 |
| OVp3-94 | 126 | Hu. polyadenylate-binding | polyadenylate-binding |
| | | PAIP1 | |
| OVp3-95 | 127 | Human TAK1-binding | Unknown |
| OVp3-97 | 128 | Hu ribosomal L27 | Hu ribosomal L27 |
| OVp3-101 | 129 | Human cytoskeletal g-actin | Unknown |
| OVp3-104 | 85 | mitochondrial; Hu. clone | Novel |
| | | 23884 | |
| OVp3-107 | 66 | Novel | Novel |
| OVp3-110 | 67 | Novel | Novel |
| OVp3-127 | 68 | Novel | Novel; 50% homology to |
| | | | human hepatoma-derived |
| | | | growth factor |

TABLE III
Further Ovarian Carcinoma Antigen Partial Sequences

| | | SEQ | |
|---|---|---|---|
| | | ID | |
| Clone No. | Sequence | NO | Homology |
| OVp2-3 | 15674.2 | 130 | Novel |
| OVp2-8 | 15679.2 | 131 | A. t BAC F21E10; ESTs |
| OVp2-26 | 20045.1 | 132 | Human I-mf; ETS |
| OVp2-33 | 20047.1 | 133 | Novel; ESTs |
| OVp2-34 | 20056.1 | 134 | BiP, GRP; ESTs |
| OVp2-45 | 17295.1 | 135 | Human Chrom 16; ESTs |
| OVp2-49 | 20209.1 | 136 | Human C90RF3 short isoform; ESTs |
| OVp2-53 | 16111.2 | 137 | Novel; ESTs |
| OVp2-61 | 22421.1 | 138 | Novel |
| OVp2-62 | 22422.1 | 139 | TNF-α stimulated ATP-binding; ESTs |
| OVp2-63 | 22423.1 | 140 | Human.DNA from phage pTELchrom. |
| | | | 16p13.3; ESTs |
| OVp2-64 | 22424.1 | 141 | Human mRNA for fibronectin; ESTs |
| OVp2-65 | 22425.1 | 142 | Novel; ESTs |
| OVp2-74 | 22428.1 | 143 | Human deoxyhypusine synthase |
| | | | mRNA; ESTs |
| OVp2-75 | 22430.1 | 144 | Novel |
| OVp2-76 | 22431.1 | 145 | Novel; ESTs |
| OVp2-77 | 22432.1 | 146 | Novel; ESTs |
| OVp2-78 | 22433.1 | 147 | Human.cDNA DKFZp586E0518; ESTs |
| OVp2-79 | 22434.1 | 148 | Human DNA cosmid U131B10 |
| OVp2-95 | 20213.1 | 149 | Hu. Protective protein; ESTs |
| OVp2-97 | 20214.1 | 150 | Plasma protein S; ESTs |
| OVp2-107 | 20216.1 | 151 | Human epiderm al carcinoma mRNA |
| | | | for E2; ESTs |
| OVp2-113 | 20496.1 | 152 | Human Histone H1'; ESTs |
| OVp2-114 | 20497.1 | 153 | Novel |
| OVp2-116 | 18077.1 | 154 | Human Pyruvate dehydrogenase kinase; |
| | | | EST |
| OVp2-119 | 18078.1 | 155 | Human KIAA0803, BAC clone; ESTs |
| OVp2-121 | 20498.1 | 156 | Murine DNA-binding, Zn Finger; ESTs |
| OVp2-122 | 18085.1 | 157 | Rat trg |
| OVp2-124 | 20500.1 | 158 | Novel; ESTs |
| OVp2-127 | 18084.1 | 159 | Human Cosmid F23149 |
| OVp2-128 | 20501.1 | 160 | Human GlcNac 1-P transferase; ESTs |
| OVp2-129 | 20502.1 | 161 | Novel; EST |
| OVp2-131 | 18573.2 | 162 | Human laminin alpha 5 chain; ESTs |
| OVp2-133 | 18574.1 | 163 | Novel |
| OVp2-134 | 18345.1 | 164 | Human tazarotene-induced gene 2; |
| | | | ESTs |
| OVp2-135 | 18575.1 | 165 | Actin-binding P57, coronin-like; ESTs |
| OVp2-139 | 18728.1 | 166 | Human clone 1033D10; includes |
| | | | BING5; ESTs |
| OVp2-141 | 18577.1 | 167 | Human cosmid F23149; EST |
| OVp2-143 | 18881.1 | 168 | c-myc proto-oncogene; ESTs |
| OVp2-144 | 18882.1 | 169 | Murine, Human nucleic acid-binding |
| | | | protein; ESTs |
| OVp2-146 | 18884.1 | 170 | Novel; ESTs |
| OVp2-147 | 18885.1 | 171 | Human B23 nucleophosmin; ESTs |
| OVp2-148 | 18886.1 | 172 | cation-dependent Human,Murine |
| | | | MP-6R; ESTs |
| OVp2-150 | 18889.1 | 173 | Human FXR1; ESTs |
| OVp2-152 | 18891.1 | 174 | Human KIAA0465; ESTs |
| OVp2-153 | 18892.1 | 175 | Human α-2macroglobin receptor assoc; |
| | | | ESTs |
| OVp2-158 | 20027.1 | 176 | Topoisomerase II; ESTs |
| OVp2-160 | 20028.1 | 177 | Novel |
| OVp2-162 | 20029.1 | 178 | Novel |
| OVp2-167 | 20035.1 | 179 | KIAA0630; ESTs |
| OVp2-169 | 20037.1 | 180 | Novel |
| OVp2-171 | 20039.1 | 181 | Novel; ESTs |
| OVp2-172 | 20072.1 | 182 | Novel |
| OVp2-174 | 20031.1 | 183 | mig-2; ESTs |
| OVp2-179 | 20040.1 | 184 | Novel; ESTs |
| OVp2-180 | 20041.1 | 185 | Antiquin; turgor protein; ESTs |
| OVp2-184 | 20044.1 | 186 | Human ribosomal P1; ESTs |
| OVp2-185 | 20057.1 | 187 | Novel; ESTs |
| OVp2-187 | 20074.1 | 188 | MuLV env |
| OVp2-188 | 20075.1 | 189 | Novel; EST |
| OVp2-189 | 20058.1 | 190 | Novel; EST |
| OVp2-190 | 20059.1 | 191 | Gonadotropin-Reg Hormone Producing; |
| | | | ESTs |
| OVp2-191 | 21502.1 | 192 | Novel; ESTs |
| OVp2-192 | 21503.1 | 193 | 18S rRNA, DNA; ESTs |
| OVp2-193 | 21504.1 | 194 | Arg-rich Nuclear Protein; ESTs |
| OVp2-194 | 21505.1 | 195 | Novel; ESTs |
| OVp2-195 | 21506.1 | 196 | Novel; ESTs |
| OVp2-196 | 21507.1 | 197 | Human Clone 406A7; ESTs |
| OVp2-197 | 21508.1 | 198 | Novel; ESTs |
| OVp2-204 | 22128.1 | 199 | Human Mitochondrial DNA; ESTs |
| OVp2-206 | 22129.1 | 200 | Human chrom. DNA for RAD23A; |
| | | | ESTs |
| OVp2-207 | 22130.1 | 201 | Human clone 24921 mRNA; ESTs |
| OVp2-208 | 22131.1 | 202 | Human 4F5rel mRNA; ESTs |
| OVp2-209 | 22133.1 | 203 | Human ribosomal pro. S6 |
| | | | kinase.SW1/SNF related; ESTs |
| OVp2-211 | 22134.1 | 204 | Human beta-glucuronidase (BG) |
| | | | mRNA; ESTs |
| OVp2-212 | 22135.1 | 205 | Human DNA for hnRNP protein A2/B1; |
| | | | ESTs |
| OVp2-215 | 22137.1 | 206 | Human translocation protein 1; ESTs |
| OVp2-216 | 22138.1 | 207 | Human chromosone X orf5; ESTs |
| OVp2-217 | 22139.1 | 208 | Human ribosomal protein S19; ESTs |
| OVp2-218 | 22140.1 | 209 | Murine mRNA for histone H3.3A; |
| | | | ESTs |
| OVp2-220 | 22141.1 | 210 | Human PAC 434P1; ESTs |
| OVp2-221 | 22142.1 | 211 | Human AKAP450.KIAA0803.Hyper- |
| | | | ion; ESTs |
| OVp2-222 | 22144.1 | 212 | Human HRFX2 mRNA, DNA binding |
| OVp2-223 | 22145.1 | 213 | Human NF-kappa-B transcrip'n factor |
| | | | p65; ESTs |
| OVp2-225 | 22146.1 | 214 | Novel |
| OVp2-226 | 22147.1 | 215 | Novel |
| OVp2-228 | 22148.1 | 216 | Novel; ESTs |
| OVp2-229 | 22149.1 | 217 | Human mitochondrial genes; ESTs |
| OVp2-230 | 22150.1 | 218 | O. cuniculus endooligopeptidase A |
| | | | related protein; ESTs |
| OVp2-232 | 22152.1 | 219 | Human clone A9A2BR11; Mu Zfr |
| OVp2-233 | 22153.1 | 220 | Human KIAA0098; Murine chaperonin |
| | | | containing TCP-1; ESTs |
| OVp2-238 | 22154.1 | 221 | human DNA seq. from PAC 93H18; |
| | | | ESTs |
| OVp2-239 | 22155.1 | 222 | Novel |
| OVp2-241 | 22156.1 | 223 | Human glutathione S-transferase |
| | | | theta 1; ESTs |
| OVp2-243 | 22157.1 | 224 | Human TCB.OIP3, pyruvate kinase; |
| | | | ESTs |
| OVp2-244 | 22158.1 | 225 | Human mRNA for KIAA0250 gene; |
| | | | EST |
| OVp2-250 | 22160.1 | 226 | Human complement component C4A |
| | | | mRNA; EST |
| OVp2-251 | 22161.1 | 227 | Novel; EST |
| OVp2-252 | 22162.1 | 228 | Novel; EST |
| OVp2-253 | 22163.1 | 229 | Human. G protein Golf alpha gene; |
| | | | ESTs |
| OVp2-254 | 22164.1 | 230 | Novel; ESTs |
| OVp2-257 | 22897.1 | 231 | Novel; ESTs |
| OVp2-258 | 22440.1 | 232 | Human chrom 16. cosmid clone |
| | | | 399H11; ESTs |
| OVp2-259 | 22441.1 | 233 | human cDNA DKFZp564B112; ESTs |
| OVp2-260 | 22898.1 | 234 | Novel; ESTs |
| OVp2-262 | 22442.1 | 235 | Human mRNA for ribosomal protein |
| | | | L31; ESTs |
| OVp2-265 | 22899.1 | 236 | Human TNF receptor mRNA; ESTs |
| OVp2-266 | 22445.1 | 237 | Human 12q13.1 PAC RPCl1-228P16 |
| OVp2-270 | 22447.1 | 238 | Hu. cDNA DKFZp586F1523; ESTs |
| OVp2-273 | 22450.1 | 239 | Homo sapiens cytochrome b-245; ESTs |
| OVp2-276 | 22451.1 | 240 | Novel; ESTs |
| OVp2-279 | 22454.1 | 241 | Novel; ESTs |
| OVp2-282 | 22903.1 | 242 | Novel; ESTs |
| OVp2-283 | 22904.1 | 243 | Human KIAA9001 mRNA,RING3; |
| | | | ESTs |
| OVp2-284 | 22905.1 | 244 | Novel; ESTs |
| OVp2-285 | 22906.1 | 245 | Novel; ESTs |
| OVp2-287 | 22907.1 | 246 | Novel |
| OVp3-15 | 20048.1 | 247 | JM26; ESTs |
| OVp3-27 | 20049.1 | 248 | mult. Human BAC; Line1; ESTs |
| OVp3-42 | 20050.1 | 249 | Tyrosine phosphatase; ESTs |
| OVp3-58 | 20052.1 | 250 | Novel; ESTs |
| OVp3-61 | 20060.1 | 251 | Novel; ESTs |
| OVp3-73 | 20064.1 | 252 | glypican-4 |
| OVp3-74 | 20065.1 | 253 | Novel; ESTs |
| OVp3-78 | 20069.1 | 254 | Novel |
| OVp3-80 | 20053.1 | 255 | MLN 50 RNA; EST |
| OVp3-89 | 20217.1 | 256 | Novel |
| OVp3-108 | 20222.1 | 257 | Human parathymosin; ESTs |
| OVp3-109 | 20223.1 | 258 | Human eryth/α-adductin; ESTs |
| OVp3-114 | 18080.1 | 259 | Novel |
| OVp3-115 | 20225.1 | 260 | Human JM26; ESTs |
| OVp3-116 | 20226.1 | 261 | Novel; ESTs |
| OVp3-120 | 20503.1 | 262 | Human KIAA0875; ESTs |
| OVp3-121 | 20227.1 | 263 | Human Guanine-binding; ESTs |
| OVp3-122 | 20228.1 | 264 | Novel; ESTs |
| OVp3-123 | 18086.1 | 265 | Novel |
| OVp3-124 | 18087.1 | 266 | Human transposon L1.2; ESTs |
| OVp3-127 | 18089.1 | 267 | low sim. to Mu.Hepatoma GF; ESTs |
| OVp3-129 | 20504.1 | 268 | Human β spectrin (actin-binding); ESTs |
| OVp3-130 | 18347.1 | 269 | BAC GS083B20; Line1, p150; ESTs |
| OVp3-131 | 18348.1 | 270 | glutathione S-transferase; ESTs |
| OVp3-132 | 18349.1 | 271 | Human mRNA KIAA0710; EST |
| OVp3-136 | 20506.1 | 272 | Novel; ESTs |
| OVp3-137 | 18731.1 | 273 | Novel; ESTs |
| OVp3-142 | 20508.1 | 274 | Novel |
| OVp3-144 | 18735.1 | 275 | Novel; EST |
| OVp3-147 | 18738.1 | 276 | KIAA0941,PGEMEX; ESTs |
| OVp3-148 | 20510.1 | 277 | Polyubiquitin; ESTs |
| OVp3-149 | 18894.1 | 278 | Novel; ESTs |
| OVp3-150 | 18895.1 | 279 | Human SWI/SNF; ESTs |
| OVp4-1 | 20017.1 | 280 | Novel; EST |
| OVp4-2 | 20018.1 | 281 | Human KIAA0241; ESTs |
| OVp4-4 | 20019.1 | 282 | Novel; ESTs |
| OVp4-6 | 20020.1 | 283 | Novel; ESTs |
| OVp4-7 | 20021.1 | 284 | laminin-binding; ESTs |
| OVp4-8 | 20022.1 | 285 | okadaic-acid-inducible; ESTs |
| OVp4-10 | 20023.1 | 286 | MAC25; ESTs |
| OVp4-13 | 20054.1 | 287 | Novel; ESTs |
| OVp4-14 | 20055.1 | 288 | Novel |
| OVp4-15 | 20076.1 | 289 | Human HSP; ESTs |
| OVp4-16 | 20077.1 | 290 | Clathrin; σ3A (AP-3 complex); ESTs |
| OVp4-18 | 20078.1 | 291 | Novel; ESTs |
| OVp4-20 | 20070.1 | 292 | Novel |
| OVp4-22 | 20229.1 | 293 | Human β-Catenin; ESTs |
| OVp4-22A | 20511.1 | 294 | llPPL1(51C); DNA repair; ESTs |
| OVp4-23 | 20230.1 | 295 | Transcrp'n Fact. AP-1, JUN A, C-JUN; |
| | | | ESTs |
| OVp4-24 | 20231.1 | 296 | Novel; ESTs |
| OVp4-25 | 20512.1 | 297 | Novel; ESTs |
| OVp4-26 | 20232.1 | 298 | ribosomal P0; ESTs |
| OVp4-26A | 20538.1 | 299 | Novel; ESTs |
| OVp4-28 | 20234.1 | 300 | Transcrp'n Factor S-II; ESTs |
| OVp4-29 | 20235.1 | 301 | Novel; ESTs |
| OVp4-30 | 20236.1 | 302 | Human AHNAK; neuroblast diff'n.; |
| | | | ESTs |
| OVp4-31 | 20237.1 | 303 | CD81(TAPA-1) cell surface; ESTs |
| OVp4-33 | 20545.1 | 304 | Novel; ESTs |
| OVp4-34 | 20546.1 | 305 | Novel |
| OVp4-35 | 20547.1 | 306 | Novel; ESTs |
| OVp4-36 | 21510.1 | 307 | Novel |
| OVp4-37 | 20548.1 | 308 | Novel; ESTs |
| OVp4-38 | 22166.1 | 309 | Novel; ESTs |
| OVp4-40 | 20549.1 | 310 | Human Profilin; EST |
| OVp4-43 | 20551.1 | 311 | Novel |
| OVp4-44 | 20552.1 | 312 | Human 30M3; ESTs |
| OVp4-45 | 20553.1 | 313 | Chrom. 14 specific cosmid |
| OVp4-46 | 20554.1 | 314 | Novel; ESTs |
| OVp4-47 | 20555.1 | 315 | EF-1 α; ESTs |
| OVp4-48 | 20556.1 | 316 | B-CAM mRNA; EST |
| OVp4-49 | 20557.1 | 317 | Novel |
| OVp4-51 | 20559.1 | 318 | Human N-cadherin; ESTs |
| OVp4-52 | 20560.1 | 319 | Human p16INK4/MTS1; ESTs |
| OVp4-53 | 20561.1 | 320 | Human RNA helicase |
| OVp4-54 | 20562.1 | 321 | Murine Fibronectin; ESTs |
| OVp4-55 | 20778.1 | 322 | RibosomalS6(kinase substrate); ESTs |
| OVp4-56 | 20779.1 | 323 | Novel; ESTs |
| OVp4-58 | 20781.1 | 324 | CPG island DNA; EST |
| OVp4-59 | 20782.1 | 325 | Human KIAA 0241; ESTs |
| OVp4-61 | 20784.1 | 326 | Human PAC; EST |
| OVp4-62 | 20785.1 | 327 | Na/H reg. factor; ESTs |
| OVp4-63 | 20786.1 | 328 | Ferritin Heavy Chain; ESTs |
| OVp4-64 | 20787.1 | 329 | MHC -1 H2; ESTs |
| OVp4-66 | 20789.1 | 330 | RNA Helicase-related; ESTs |
| OVp4-70 | 20793.1 | 331 | BC-2protein RNA; ESTs |
| OVp4-72 | 20795.1 | 332 | Phosphorylase Kinase; ESTs |
| OVp4-73 | 20796.1 | 333 | Novel |
| OVp4-74 | 20797.1 | 334 | Human clone 327J16; ESTs |

### Example 2

### Analysis of cDNA Expression Using Microarray Technology and Real Time PCR

Several of the clones identified in Example 1 were further studied to determine their full-length DNA and protein sequences (see Table IV). Expression profiles for a selection of these clones were also determined using microarray analysis (see Table V) and Real Time PCR (see Table VI).

TABLE IV
Full Length DNA and Protein

| Clone Identification | DNA: SEQ ID NO | Protein: SEQ ID NO |
|---|---|---|
| O55E/OVp2-1 | 335 | 346 |
| (SEQ ID NO: 86) | | |
| O56E/OVp2-2 | 336 | |
| (SEQ ID NO: 19) | | |
| O57E/OVp2-4 | 337 | 347 |
| (SEQ ID NO: 87) | | |
| O58E/OVp2-56 | 338 | 348 |
| (SEQ ID NO: 34) | | |
| O53E/OVp2-30 | 339 | |
| O59E/OVp2-88 | 340 | 349 |
| (SEQ ID NO: 42) | | |
| O60E/OVp2-94 | 341 | 350 |
| (SEQ ID NO: 45) | | |
| OVp2-28 | 342 | 351 |
| (SEQ ID NO: 26) | | |
| OVp2-244 | 343 | 352 |
| (SEQ ID NO: 225) | | |
| OVp2-285 | 344 | |
| (SEQ ID NO: 245) | | |
| OVp4-2 | 345 | 353 |
| (SEQ ID NO: 281) | | |

TABLE V
Microarray Expression

| | Ratio | | |
|---|---|---|---|
| Clone | Tumor:Normal | Tumors | Normal Tissues |
| O55E/OVp2-1 | 1.38 | OE in | SE: trachea, pituitary, thymus |
| | | 8/34 | |
| O65E/OVp2-2 | 1.28 | HOE in | SE: thyroid, liver, trachea, |
| | | 6/34 | aorta, pancreas, pituitary, |
| | | | peritoneum, thymus |
| O57E/OVp2-4 | 1.42 | HOE in | SE: thymus |
| | | 1/34 | |
| O58E/OVp2-56 | 1.33 | HOE in | SE: most normal tissue |
| | | 34/34 | |
| O53E/OVp2-30 | 1.25 | OE in | SE: trachea, spinal cord, heart |
| | | 14/34 | ovary, stomach, pituitary, |
| | | | peritoneum, brain, thymus |
| O59E/OVp2-88 | 1.00 | OE in | SE: thyroid, trachea, skeletal |
| | | 12/34 | muscle, sm. intestine, kidney, |
| | | | heart, spleen, adrenal, pitui- |
| | | | tary, peritoneum |
| O60E/OVp2-94 | 1.13 | HOE in | SE: Trachea, thyroid, skel. |
| | | 9/34 | muscle, heart, spleen, brain, |
| | | | spinal cord |
| OVp2-28 | 1.12 | OE in | SE: trachea, pituitary |
| | | 5/30 | |
| OVp2-244 | 1.23 | OE in | SE: trachea, colon, bone mar- |
| | | 10/30 | row, heart, pituitary, perito- |
| | | | neum, bone, brain |
| OVp2-285 | 1.3628 | OE in | SE: trachea, colon, esophagus, |
| | | 6/30 | pituitary, peritoneum |
| OVp4-2 | 1.30 | HOE in | SE: trachea, aorta, pituitary, |
| | | 4/30 | peritoneum, thymus |
| | | OE in | |
| | | 7/30 | |

OE = everexpressed; HOE = highly overexpressed; SE = some expression.

Real time PCR analysis was performed on clones O55E (OVp2-1), O57E (OVp2-4), and O58E (OVp2-56).

Clone O55E was expressed eleven times higher in one tumor sample when compared to normal tissues. In addition, it was over-expressed in all other tumor samples tested, as well as showing some expression in normal trachea, pituitary and thymus.

Clone O57E was overexpressed in all tumor samples tested. Levels expressed in bone marrow were comparable to that seen in tumors, while expression levels were higher in normal thymus.

Clone O58E was expressed at least two times higher in 8/15 tumor samples tested than was found in normal tissues, with 4/15 samples showing expression levels 3-8 times higher than normal. Normal liver and ureter showed O58E levels comparable to some of the tumors which expressed lower levels of O58E.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. A method for detecting the presence of ovarian cancer in a patient, comprising the steps of:
(a) obtaining a biological sample from a patient;
(b) contacting the biological sample with an oligonucleotide that hybridizes to a sequence set forth in any one of SEQ ID NO: 335-345 under highly stringent conditions;
(c) detecting in the sample an amount of a polynucleotide that hybridizes to the oligonucleotide; and
(d) comparing the amount of polynucleotide that hybridizes to the oligonucleotide to a predetermined cut-off value, and therefrom detecting the presence of ovarian cancer in the patient.

2. The method of claim 1, wherein said detecting in the sample an amount of a polynucleotide that hybridizes to the oligonucleotide is performed by a polymerase chain reaction.

3. The method of claim 1, wherein the biological sample is selected from the group consisting of serum and ovarian tissue.

4. An oligonucleotide useful in the detection of ovarian cancer in a patient, wherein said oligonucleotide hybridizes to a sequence set forth in any one of SEQ ID NO: 335-345 under highly stringent conditions.

5. A diagnostic kit comprising at least one oligonucleotide according to claim 4.

6. A method for detecting the presence of a cancer in a patient, comprising the steps of:
(a) obtaining a biological sample from a patient;
(b) contacting the biological sample with a binding agent that binds to a polypeptide selected from the group consisting of:
(i) a polypeptide encoded by a polynucleotide sequence set forth in any one of SEQ ID NO: 335-345;
(ii) a sequence having at least 90% identity to said polypeptide;
(iii) a sequence having at least 95% identity to said polypeptide;
(c) detecting in the sample an amount of polypeptide that binds to the binding agent; and
(d) comparing the amount of polypeptide to a predetermined cut-off value and therefrom detecting the presence of a cancer in the patient.

7. A method for stimulating and/or expanding T cells specific for an ovarian tumor protein, comprising contacting T cells with at least one component selected from the group consisting of:
(a) a polypeptide sequence selected from the group consisting of:
(i) a polypeptide encoded by a polynucleotide sequence set forth in any one of SEQ ID NO: 335-345;
(ii) a sequence having at least 90% identity to said polypeptide;
(iii) a sequence having at least 95% identity to said polypeptide;
(b) a polynucleotide selected from the group consisting of:
(i) a sequence set forth in any one of SEQ ID NO: 335-345;
(ii) a complement of a sequence set forth in any one of SEQ ID NO: 335-345;
(iii) a sequence consisting of at least 20 contiguous residues of a sequence set forth in any one of SEQ ID NO: 335-345;
(iv) a sequence that hybridizes to a sequence set forth in any one of SEQ ID NO: 335-345, under highly stringent conditions;
(v) a sequence having at least 90% identity to a sequence set forth in any one of SEQ ID NO: 335-345; and
(vi) a sequence having at least 95% identity to a sequence set forth in any one of SEQ ID NO: 335-345.

8. An isolated T cell population, comprising T cells prepared according to the method of claim 7.

9. A composition comprising a first component selected from the group consisting of physiologically acceptable carriers and immunostimulants, and a second component selected from the group consisting of:
(a) a polypeptide sequence selected from the group consisting of:
(i) a polypeptide encoded by a polynucleotide sequence set forth in any one of SEQ ID NO: 335-345;
(ii) a sequence having at least 90% identity to said polypeptide;
(iii) a sequence having at least 95% identity to said polypeptide;
(b) a polynucleotide sequence selected from the group consisting of:
(i) a sequence set forth in any one of SEQ ID NO: 335-345;
(ii) a complement of a sequence set forth in any one of SEQ ID NO: 335-345;
(iii) a sequence consisting of at least 20 contiguous residues of a sequence set forth in any one of SEQ ID NO: 335-345;
(iv) a sequence that hybridizes to a sequence set forth in any one of SEQ ID NO: 335-345 under highly stringent conditions;
(v) a sequence having at least 95% identity to a sequence set forth in any one of SEQ ID NO: 335-345;
(vi) a degenerate variant of a sequence set forth in any one of SEQ ID NO: 335-345;
(c) a T cell population according to claim 8; and
(d) antigen presenting cells that express a polypeptide selected from the group consisting of:
(i) a polypeptide encoded by a polynucleotide sequence set forth in any one of SEQ ID NO: 335-345.
(ii) a sequence having at least 90% identity to said polypeptide; and
(iii) a sequence having at least 95% identity to said polypeptide.
